SEQUENCE LISTING (4 sequences)

SEQ ID NO: 1 - DNA, 21 bp, Homo sapiens
ucgagaaucuaaacuaacutt

SEQ ID NO: 2 - DNA, 21 bp, Homo sapiens
aguuaguuuagauucucgatt

SEQ ID NO: 3 - DNA, 21 bp, Photinus pyralis
cuuacgcugaguacuucgatt

SEQ ID NO: 4 - DNA, 21 bp, Photinus pyralis
ucgaaguacucagcguaagtt

(19) United States
(12) Patent Application Publication
(10) Pub. No.: US 2013/0072543 A1
(43) Pub. Date: Mar. 21, 2013

(54) Compounds and Compositions for Nucleic Acid Formulation and Delivery

(71) Applicants: John Frederick Boylan, Bedminster, NJ (US); Wei He, Livingston, NJ (US)

(72) Inventors: Michael J. Bennett, Monrovia, CA (US); John Frederick Boylan, Bedminster, NJ (US); Wei He, Livingston, NJ (US)

(73) Assignee: ARROWHEAD RESEARCH CORPORATION, Pasadena, CA (US)

(21) Appl. No.: 13/541,891

(22) Filed: Jul. 5, 2012

Related U.S. Application Data
(60) Provisional application No. 61/507,145, filed on Jul. 13, 2011.

(51) Int. Cl. A61K 47/22

(52) U.S. Cl. CPC A61K 47/22; Y10S 977/773
USPC 514/44 A; 977/773

(57) ABSTRACT

The invention relates to compositions containing compounds of formula I:

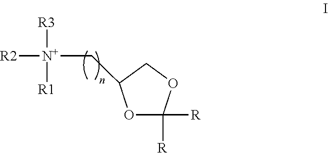

and pharmaceutically acceptable salts thereof, wherein R, R¹, R², R³, and n are defined in the detailed description and claims. In addition, the present invention relates to novel formulations containing compounds of formula I for improved delivery of nucleic acids such as siRNA to the cytoplasm of target cells. In particular embodiments these formulations comprise compounds of formula I, phospholipids, cholesterol, and pegylated lipids. The present invention also relates to methods of manufacturing and using such compounds and compositions.

### CROSS-REFERENCE TO RELATED APPLICATIONS

This application claims the benefit of U.S. provisional application Ser. No. 61/507,145, filed 13 Jul. 2011.

### FIELD OF THE INVENTION

The present invention relates to compounds and compositions used to formulate nucleic acids or oligonucleotides for delivery to cells to inhibit or prevent the expression of target genes. In particular embodiments, the present invention relates to compounds and compositions used to formulate double stranded ribonucleic acids or siRNA molecules for delivery to cells to induce RNA interference (RNAi) of target mRNA molecules, thereby inhibiting the expression of target gene products.

### BACKGROUND OF THE INVENTION

RNA interference is a well-known process in which the translation of messenger RNA (mRNA) into protein is interfered with by the association or binding of complementary or partially complementary oligonucleotides such as small interfering RNA (siRNA), short hairpin RNA(shRNA), micro RNA (miRNA), or antisense oligonucleotides. siRNAs are double-stranded RNA molecules, usually ranging from 19-25 nucleotides in length that associate with a set of proteins in the cytoplasm known as RISC (RNA-induced silencing complex). RISC ultimately separates the double stranded siRNA allowing one strand to bind or associate with a complementary or partially complementary portion of an mRNA molecule after which the mRNA is destroyed by RISC or otherwise prevented from being translated- consequently suppressing the expression of the encoded protein or gene product.

One of the problems in using nucleic acids such as siRNA in therapeutic applications (especially for systemic administration in humans) has been in delivering the nucleic acids to: (1) particular target tissues or cell types and (2) to the cytoplasm of those cells (i.e., where the mRNA is present and translated into protein). Part of the delivery problem is based on the fact that nucleic acids are negatively charged and easily degraded (especially if unmodified), efficiently filtered by the kidney, and cannot be easily transported to the cytoplasm of the cells by themselves. Thus, a significant amount of research has focused on solving the delivery problem with various carriers and formulations including liposomes, micelles, peptides, polymers, conjugates and aptamers (Ling et al. “Advances in Systemic siRNA Delivery,” Drugs Future 2009 34(9):721). Some of the more promising delivery vehicles have involved the use of lipidic systems—including lipid nanoparticles (Wu et al. “Lipidic Systems for In Vivo siRNA Delivery”, AAPS J. 2009 11(4):639-652; Hope et al. “Improved Amino Lipids And Methods For the Delivery of Nucleic Acids” International Patent Application Publication No. WO 2010/042877. However, clinical trials using lipid-based nanoparticles (LNPs) have been somewhat limited by safety concerns, antibody opsonization and phagocytosis, and an inability to deliver nucleic acids, such as siRNA, to organs other than the liver and lung after intravenous administration. Thus, a need remains for further improved carriers and formulations including lipid-based nucleic acid delivery systems capable of safely and efficiently delivering nucleic acids such as siRNA to particular target cells and to the cytoplasm of such cells, while avoiding or reducing reticuloendothelial clearance and/or opsonization.

### SUMMARY OF THE INVENTION

The invention relates to compositions containing a compound of formula I:

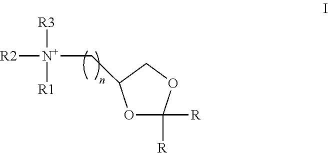

and pharmaceutically acceptable salts thereof, wherein R, R¹, R², R³, and n are defined in the detailed description and claims. In addition, the present invention relates to novel formulations containing compounds of formula I for improved delivery of nucleic acids such as siRNA to the cytoplasm of target cells. In particular embodiments these compositions or formulations comprise a compound of claim 1, a phospholipid, cholesterol, a pegylated lipid, and a polynucleotide. The present invention also relates to methods of manufacturing and using such compounds and compositions.

The compounds of formula I are useful as components in particular lipid nanoparticle compositions or formulations which improve the delivery of nucleic acids such as siRNA to the cytoplasm of target cells. In particular embodiments, the present invention relates to lipid nanoparticle formulations containing the compounds of formula I which are useful in delivering siRNA to the cytoplasm of target cells to inhibit the expression of certain target proteins through RNA interference. In more particular embodiments, the present invention relates to novel lipids and novel lipid compositions containing the compounds of formula I that effectively deliver siRNA to tumor cells and other cell types dependent on the expression of endocytosis genes. The invention also provides novel compositions containing the compounds of formula I and methods for treating pathological conditions and diseases caused by the expression of different genes such as in proliferative disorders like cancer and inflammation. Such compositions and formulations are more efficacious and demonstrate improved knockdown capability compared to similar formulations lacking the compounds of formula I.

### BRIEF DESCRIPTION OF THE DRAWINGS

FIG. 1. Bar graph illustrating KIF11 mRNA knockdown in a suspension of Molm13 cells after 24 hrs of treatment with various LNP formulations containing KIF11 siRNA (as disclosed in Table 1). KIF11 mRNA knockdown is measured relative to cellular 18S ribosomal ribonucleic acid (rRNA) content. As indicated, the XTC2-based LNPs (formulation nos. 23A-G as disclosed in Table 1) were able to effectively deliver KIF11 siRNA into Molm13 cells and knock down KIF11 mRNA.

FIG. 2. Structures for XTC2 and CDM-XTC-2. illustrating the difference in structure between the compound of example 1 (referred to herein as “XTC2” or “DLin-KC2-DMA”) and the compound of example 4 (referred to herein as “DCM-XTC2” or “DLin-KC2-CIMDMA”).

FIG. 3. Differential scanning calorimetry (DSC) curves of siRNA LNPs in the absence or presence of DCM-XTC2 (the compound of example 4). As indicated, the presence of DCM-XTC2 (in formulation no. 67A as disclosed in Table 1) significantly changed the physical properties of the LNPs (i.e., phase transition behavior was observed with the alkylated lipid which is consistent with a change in the physical properties of the LNPs) when compared to similar formulations that do not contain this lipid (67B and 67C as disclosed in Table 1).

FIG. 4. Bar graph illustrating KIF11 mRNA knockdown in a suspension Molm13 cells after 24 hrs of treatment with various LNP formulations containing KIF11 siRNA (formulation nos. 85A-85H, 23B, and 67A as disclosed in Table 1).

FIGS. 5a and 5b. Bar graphs illustrating KIF11 mRNA knockdown in a suspension PC3 (FIG. 5a) and Molm13 (FIG. 5b) cells after 24 hrs of treatment with various LNP formulations containing KIF11 siRNA (formulation nos. 23C and 123A-1231).

FIG. 6. Chemical structures of various XTC derivatives with differences in the head group (from top to bottom, examples 3, 2, and 4 respectively) indicating increased mRNA knockdown ability as indicated by the arrow.

FIG. 7. Bar graph illustrating KIF11 mRNA knockdown in a suspension Molm13 cells after 24 hrs of treatment with various LNP formulations containing KIF11 siRNA (formulation nos. 23B and 95A-95I as disclosed in Table 1).

FIG. 8. Bar graph illustrating KIF11 mRNA knockdown in different suspension leukemia cell lines (HEL, K562, KG1, MV4-11, THP-1, and Molm13) after 24 hrs of treatment with LNP formulation nos. 67A and 67B containing KIF11 siRNA (as disclosed in Table 1). As indicated, formulation no. 67A (containing the compound of example 4 referred to as DCM-XTC2 or DLin-KC2-CIMDMA) had a greater knockdown effect than formulation no. 67B (which only contained the compound of example 1 referred to as XTC2 or DLin-KC2-DMA).

FIG. 9. H&E staining of cross-sections of a variety of mouse tissues treated with control luciferase siRNA using formulation no. 911 (disclosed in Table 1, XTC2 51.9:DPPC 6.9: Chol 33.2: PEG-c-DMG 5.8: XTC2-DCM 2.2; P/N 1.92; 151 nm). Mice received a single IV administration of siRNA (4 mg/kg) and tissues were harvested 48 hr post-dose.

FIG. 10. H&E staining of cross-sections of a variety of mouse tissues treated with KIF11 siRNA using formulation no. 91D (disclosed in Table 1, XTC2 51.9:DPPC 6.9: Chol 33.2: PEG-c-DMG 5.8: XTC2-DCM 2.2; N/P 1.92; 149 nm). Mice received a single IV administration of siRNA (4 mg/kg) and tissues were harvested 48 hr post-dose. Arrows indicate arrested mitotic spindles resulting from KIF11 knockdown

FIG. 11. H&E staining of cross-sections of a variety of mouse tissues treated with KIF11 siRNA using formulation no. 91G (disclosed in Table 1, XTC2 51.9:DPPC 6.9: Chol 33.2: PEG-c-DMG 5.8: XTC2-DCM 2.2; N/P 1.92; 199 nm; HA/siRNA ratio 0.5). Mice received a single IV administration of siRNA (4 mg/kg) and tissues were harvested 48 hr post-dose. Arrows indicate arrested mitotic spindles resulting from KIF11 knockdown.

### DETAILED DESCRIPTION OF THE INVENTION

Unless otherwise indicated, the following specific terms and phrases used in the description and claims are defined as follows:

The term “moiety” refers to an atom or group of chemically bonded atoms that is attached to another atom or molecule by one or more chemical bonds thereby forming part of a molecule. For example, the variables R, R¹, R², and R³ of formula I refer to moieties that are attached to the core structure of formula I by a covalent bond.

In reference to a particular moiety with one or more hydrogen atoms, the term “substituted” refers to the fact that at least one of the hydrogen atoms of that moiety is replaced by another substituent or moiety. For example, the term “lower alkyl substituted by halogen” refers to the fact that one or more hydrogen atoms of a lower alkyl (as defined below) is replaced by one or more halogen moieties (e.g., trichloromethyl, dichloromethyl, chloromethyl, chloromethyl, etc.).

The term “optionally substituted” refers to the fact that one or more hydrogen atoms of a moiety (with one or more hydrogen atoms) can be, but does not necessarily have to be, substituted with another substituent.

The term “halogen” refers to a moiety of fluoro, chloro, bromo or iodo.

Unless otherwise indicated, the term “hydrogen” or “hydro” refers to the moiety of a hydrogen atom (—H) and not H₂.

The term “alkyl” refers to an aliphatic straight-chain or branched-chain saturated hydrocarbon moiety having 1 to 22 carbon atoms.

The term “lower alkyl” refers to an alkyl moiety having 1 to 7 carbon atoms. In particular embodiments the lower alkyl has 1 to 4 carbon atoms and in other particular embodiments the lower alkyl has 1 to 3 carbon atoms. Examples of lower alkyls include methyl, ethyl, propyl, isopropyl, butyl, isobutyl, sec-butyl and tert-butyl.

The term “alkenyl” refers to an aliphatic straight-chain or branched-chain hydrocarbon moiety having 1 to 22 carbon atoms containing one or more carbon-carbon double bonds.

The term “lower alkenyl” refers to an alkenyl moiety having 1 to 7 carbon atoms. In particular embodiments the lower alkenyl has 1 to 4 carbon atoms and in other particular embodiments the lower alkenyl has 1 to 3 carbon atoms. Examples of lower alkenyls include allyl (2-pentenyl), butenyl, and pentenyl.

The term “alkynyl” refers to an aliphatic straight-chain or branched-chain hydrocarbon moiety having 1 to 22 carbon atoms containing one or more carbon-carbon triple bonds.

The term “lower alkynyl” refers to an alkynyl moiety having 1 to 7 carbon atoms. In particular embodiments the lower alkynyl has 1 to 4 carbon atoms and in other particular embodiments the lower alkynyl has 1 to 3 carbon atoms. Examples of lower alkynyls include 2-propynyl and 3-butynyl.

The term “alkoxy” refers to the moiety —O—R, wherein R is alkyl as defined previously.

The term “lower alkoxy” refers to the moiety —O—R, wherein R is lower alkyl as defined previously. Examples of lower alkoxy moieties include methoxy, ethoxy, n-propoxy, isopropoxy, n-butoxy, isobutoxy, sec-butoxy and tert-butoxy.

The term “pegylated lipid” refers to a lipid containing one or more polyethylene glycol units or moieties. Examples include, but are not limited to, R-3-[(ω-methoxy-poly(ethyleneglycol)₂₀₀₀) carbamoyl]-1,2-dimyristyloxy-propyl-3-amine, and 1,2-distearoyl-sn-glycero-3-phospho-ethanol amine-N-[methoxy(polyethylene glycol)-2000].

The term “polynucleotide” refers to DNA- or RNA-based nucleic acid polymers with unmodified or modified bases, sugars, and/or phosphate groups. Examples include, but are not limited to, antisense oligonucleotides, double-stranded DNA or RNA, small interfering RNA (siRNA), short hairpin RNA(shRNA), micro RNA (miRNA), RNA aptamers, plasmids, and DNAzymes. In preferred embodiments such polynucleotides contain at least 13 nucleotide monomers.

Unless otherwise indicated, the term “a compound of the formula” or “a compound of formula” or “compounds of the formula” or “compounds of formula” means any compound selected from the genus of compounds as defined by the formula (including any pharmaceutically acceptable salt or ester of any such compound if not otherwise noted).

The term “pharmaceutically acceptable salts” refers to those salts which retain the biological effectiveness and properties of the free bases or free acids, which are not biologically or otherwise undesirable. The salts of the present invention may be formed by the addition of inorganic or organic bases to the acid compounds of the present invention. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins and the like.

The compounds of the present invention can be present in the form of pharmaceutically acceptable salts. The compounds of the present invention can also be solvated, i.e. hydrated. The solvation can be effected in the course of the manufacturing process or can take place i.e. as a consequence of hygroscopic properties of an initially anhydrous compound of formula I (hydration).

Compounds that have the same molecular formula but differ in the nature or sequence of bonding of their atoms or the arrangement of their atoms in space are termed “isomers.” Isomers that differ in the arrangement of their atoms in space are termed “stereoisomers.” Diastereomers are stereoisomers with opposite configuration at one or more chiral centers which are not enantiomers. Stereoisomers bearing one or more asymmetric centers that are non-superimposable mirror images of each other are termed “enantiomers.” When a compound has an asymmetric center, for example, if a carbon atom is bonded to four different groups, a pair of enantiomers is possible. An enantiomer can be characterized by the absolute configuration of its asymmetric center or centers and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “a therapeutically effective amount” of a compound means an amount of compound that is effective to prevent, alleviate or ameliorate symptoms of disease or prolong the survival of the subject being treated. Determination of a therapeutically effective amount is within the skill in the art. The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. The daily dosage can be administered as a single dose or in divided doses, or for parenteral administration, it may be given as continuous infusion.

The term “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

In detail, the present invention relates to compositions comprising the compounds of formula I:

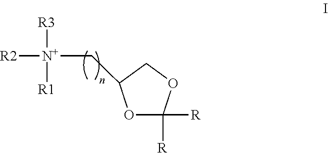

and pharmaceutically acceptable salts thereof, wherein:
R¹, R², and R³ are independently selected from the group consisting of:
   hydrogen,
   lower alkyl which is optionally substituted by hydroxy, lower alkoxy or halogen,
   lower alkenyl, and
   lower alkynyl,
   with the proviso that when at least one of R¹, R², or R³ is hydrogen, then at least one of R¹, R², or R³ is: (a) lower alkyl substituted by hydroxy, lower alkoxy or halogen, (b) lower alkenyl, or (c) lower alkynyl;
R is selected from the group consisting of:
   alkyl having 9 to 22 carbon atoms,
   alkenyl having 9 to 22 carbon atoms, and
   alkynyl having 9 to 22 carbon atoms; and
n is 1-10.

In more specific embodiments, the present invention is directed to compositions comprising compounds of formula I wherein R¹ is lower alkyl substituted by halogen.

In particular embodiments, the present invention is directed to compositions comprising compounds of formula I wherein R¹ is lower alkyl substituted by halogen; and R² and R³ are the same and independently selected from the group consisting of hydrogen and lower alkyl.

In particular embodiments, the present invention is directed to compositions comprising compounds of formula I wherein R¹ is lower alkyl substituted by halogen; R² and R³ are both lower alkyl; and R is selected from the group consisting of: nanyl, lauryl, myristyl, palmityl, stearyl, oleyl, linoleyl, arcchidyl erucyl, isostearyl, elaidyl, petroselinyl, (Z,Z)-11,13-hexadecadienyl, and (Z,Z)-7,11-hexadecadienyl.

In particular embodiments, the present invention is directed to compositions comprising compounds of formula I wherein R¹ is chloromethyl; R² and R³ are methyl; and n is 2.

In particular embodiments, the present invention is directed to compositions comprising compounds of formula I wherein R is linoleyl; R¹ is chloromethyl; R² and R³ are methyl; and n is 2.

In particular embodiments, the present invention is directed to compositions comprising compounds of formula I wherein R¹, R² and R³ are methyl; and n is 2.

In particular embodiments, the present invention is directed to compositions comprising compounds of formula I wherein R is linoleyl; R¹, R² and R³ are methyl; and n is 2.

In particular embodiments, the present invention is directed to compositions comprising compounds of formula I wherein R¹ is ethyl; R² and R³ are methyl; and n is 2.

In particular embodiments, the present invention is directed to compositions comprising compounds of formula I wherein R is linoleyl; R¹ is ethyl; R² and R³ are methyl; and n is 2.

In addition, the present invention relates to methods of manufacturing and using the compounds of formula I as well as pharmaceutical formulations containing such compounds. The compounds of formula I are useful in formulating lipid nanoparticle compositions to improve the delivery of nucleic acids such as siRNA to the cytoplasm of target cells. In particular embodiments, the present invention relates to lipid nanoparticle compositions and formulations containing the compounds of formula I which are useful in delivering siRNA to the cytoplasm of target cells to inhibit the expression of certain target proteins through RNA interference.

In more particular embodiments, the invention relates to the use of a mixture of synthetic lipid enhancers including compounds of formula I used in lipid nanoparticle compositions to facilitate the delivery of nucleic acids such as siRNA to tumor cells and other cell types. In specific embodiments, the lipid nanoparticle compositions containing compounds of formula I, show improved delivery to cells expressing Cav1, Rab13, and Rab7B which may serve as in vivo patient selection biomarkers. Furthermore, the use of synthetic lipid enhancers including compounds of formula I to synthesize lipid nanoparticle compositions to treat inflammation and proliferative disorders, like cancers, is part of the invention.

In more specific embodiments, the present invention is directed to compositions comprising a compound of formula I selected from the group consisting of:
N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethylethan-amonium iodide; and
2-(2,2-di((9Z,12Z)-octadeca-9,12-dienyl)-1,3-dioxolan-4-yl)-N-ethyl-N,N-dimethyl-ethanamonium iodide.

In other particular embodiments, the present invention is directed to lipid nanoparticle compositions and formulations comprising: a compound of formula I, a phospholipid, cholesterol, a pegylated lipid; and a polynucleotide.

In more specific embodiments, the present invention is directed to lipid nanoparticle compositions comprising:
a compound of formula I;
a phospholipid selected from the group consisting of:
   1,2-dipalmitoyl-sn-glycero-3-phosphocholine,
   1,2-distearoyl-sn-glycero-3-phosphocholine, and
   1,2-dioleoyl-sn-glycero-3-phosphocholine;
cholesterol;
a pegylated lipid compound selected from the group consisting of:
   R-3-[(ω-methoxy-poly(ethyleneglycol)₂₀₀₀)carbamoyl]-1,2-dimyristyloxy-propyl-3-amine; and
   1,2-distearoyl-sn-glycero-3-phosphoethanolamine-Ntmethoxy(polyethylene-glycol)-2000]; and
a polynucleotide.

In more specific embodiments, the present invention is directed to lipid nanoparticle compositions comprising:
a compound of formula I selected from the group consisting of:

N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethylethan-amonium iodide; and
2-(2,2-di((9Z,12Z)-octadeca-9,12-dienyl)-1,3-dioxolan-4-yl)-N-ethyl-N,N-dimethyl-ethanamonium iodide;
a phospholipid selected from the group consisting of:
   1,2-dipalmitoyl-sn-glycero-3-phosphocholine,
   1,2-distearoyl-sn-glycero-3-phosphocholine, and
   1,2-dioleoyl-sn-glycero-3-phosphocholine;
cholesterol;
a pegylated lipid compound selected from the group consisting of:
   R-3-[(ω-methoxy-poly(ethyleneglycol)₂₀₀₀)carbamoyl]-1,2-dimyristyloxy-propyl-3-amine; and
   1,2-distearoyl-sn-glycero-3-phosphoethanolamine-N4methoxy(polyethylene-glycol)-2000]; and
a polynucleotide.

The present invention is also directed to any of the lipid nanoparticle compositions disclosed herein further comprising a cationic lipid such as, but not limited to, 2-(2,2-di((9Z,12Z)-octadeca-9,12-dienyl)-1,3-dioxolan-4-yl)-N,N-dimethylethanamine as shown in example 1.

In more specific embodiments, the present invention is directed to lipid nanoparticle compositions comprising:
a compound of formula I selected from the group consisting of:
   N-(chloromethyl)-2-(2,2-di((9Z,12Z)-octadeca-9,12-dienyl)-1,3-dioxolan-4-yl)-N,N-dimethylethanamonium chloride;
   2-(2,2-di((9Z,12Z)-octadeca-9,12-dienyl)-1,3-dioxolan-4-yl)-N,N,N-trimethylethan-amonium iodide; and
   2-(2,2-di((9Z,12Z)-octadeca-9,12-dienyl)-1,3-dioxolan-4-yl)-N-ethyl-N,N-dimethyl-ethanamonium iodide;
   a phospholipid selected from the group consisting of:
      1,2-dipalmitoyl-sn-glycero-3-phosphocholine,
      1,2-distearoyl-sn-glycero-3-phosphocholine, and
      1,2-dioleoyl-sn-glycero-3-phosphocholine;
cholesterol;
a pegylated lipid compound selected from the group consisting of:

R-3-[(ω-methoxy-poly(ethyleneglycol)₂₀₀₀)carbamoyl]-1,2-dimyristyloxy-propyl-3-amine; and
1,2-distearoyl-sn-glycero-3-phosphoethanolamine-N-[methoxy(polyethylene-glycol)-2000];
a polynucleotide; and
2-(2,2-di((9Z,12Z)-octadeca-9,12-dienyl)-1,3-dioxolan-4-yl)-N,N-dimethylethanamine.

In other embodiments, the present invention is directed to any of the lipid nanoparticle compositions disclosed herein, wherein the compound of formula I is 2-(2,2-di((9Z,12Z)-octadeca-9,12-dienyl)-1,3-dioxolan-4-yl)-N-ethyl-N,N-dimethylethanamonium iodide as shown in example 2.

In other embodiments, the present invention is directed to any of the lipid nanoparticle compositions disclosed herein, wherein the compound of formula I is 2-(2,2-di((9Z,12Z)-octadeca-9,12-dienyl)-1,3-dioxolan-4-yl)-N,N,N-trimethylethanamonium iodide as shown in example 3.

In other embodiments, the present invention is directed to any of the lipid nanoparticle compositions disclosed herein, wherein the compound of formula I is N-(chloromethyl)-2-(2,2-di((9Z,12Z)-octadeca-9,12-dienyl)-1,3-dioxolan-4-yl)-N,N-dimethylethanamonium chloride as shown in example 4.

In other embodiments, the present invention is directed to any of the lipid nanoparticle compositions disclosed herein, wherein the phospholipid is 1,2-dipalmitoyl-sn-glycero-3-phosphocholine as shown below:

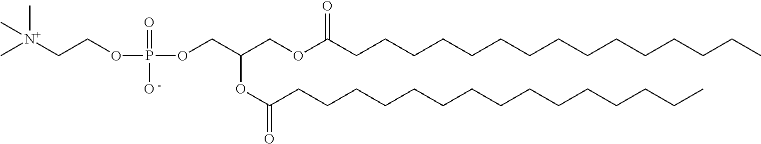 [SMILES: CCCCCCCCCCCCCCCC(=O)OCC(COP(=O)(O)OCC[N+](C)(C)C)OC(=O)CCCCCCCCCCCCCCC]

In other embodiments, the present invention is directed to any of the lipid nanoparticle compositions disclosed herein, wherein the phospholipid is 1,2-distearoyl-sn-glycero-3-phosphocholine as shown below:

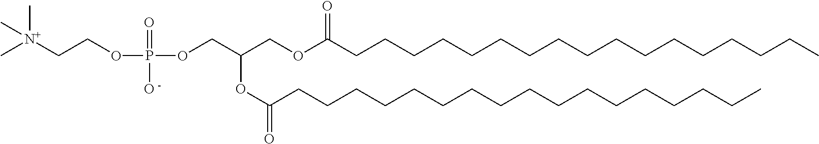 [SMILES: CCCCCCCCCCCCCCCCCC(=O)OCC(COP(=O)(O)OCC[N+](C)(C)C)OC(=O)CCCCCCCCCCCCCCCCC]

In other embodiments, the present invention is directed to any of the lipid nanoparticle compositions disclosed herein, wherein the phospholipid is 1,2-dioleoyl-sn-glycero-3-phosphocholine as shown below:

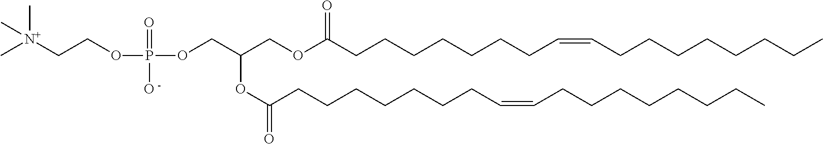 [SMILES: CCCCCCCC/C=C\CCCCCCCC(=O)OCC(COP(=O)(O)OCC[N+](C)(C)C)OC(=O)CCCCCCC/C=C\CCCCCCCC]

In other embodiments, the present invention is directed to any of the foregoing lipid nanoparticle compositions disclosed herein, wherein the pegylated lipid compound is R-3-[(ω-methoxy-poly(ethyleneglycol)₂₀₀₀)carbamoyl]-1,2-dimyristyloxy-propyl-3 -amine as shown below:

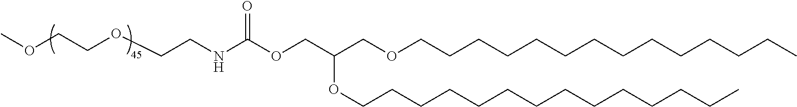

In other embodiments, the present invention is directed to any of the foregoing lipid nanoparticle compositions disclosed herein, wherein the pegylated lipid compound is 1,2-distearoyl-sn-glycero-3-phosphoethanolamine-N-[methoxy(polyethylene glycol)-2000] as shown below:

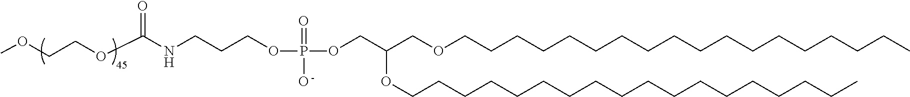

In other embodiments, the present invention is directed to any of the lipid nanoparticle compositions disclosed herein, wherein the polynucleotide is a small interfering RNA (siRNA), short hairpin RNA(shRNA), micro RNA (miRNA), or antisense oligonucleotide.

In particular embodiments the polynucleotide is a siRNA molecule, and in more particular embodiments the siRNA molecule targets the expression of KIF11.

In more specific embodiments, the present invention is directed to lipid nanoparticle compositions comprising:
(1) 2-(2,2-di((9Z,12Z)-octadeca-9,12-dienyl)-1,3-dioxolan-4-yl)-N,N-dimethylethanamine in a molar ratio in the range of about 45-55;
(2) N-(chloromethyl)-2-(2,2-di((9Z,12Z)-octadeca-9,12-dienyl)-1,3-dioxolan-4-yl)-N,N-dimethylethanamonium chloride in a molar ratio in the range of about 1.5-9;
(3) 1,2-dipalmitoyl-sn-glycero-3-phosphocholine in a molar ratio in the range of about 6-8;
(4) cholesterol in a molar ratio in the range of about 33-35; and
(5) R-3-[(ω-methoxy-poly(ethyleneglycol)₂₀₀₀)carbamoyl]-1,2-dimyristyloxy-propyl-3-amine in a molar ratio in the range of about 0.5-6.

In more specific embodiments, the present invention is directed to lipid nanoparticle compositions comprising:
(1) 2-(2,2-di((9Z,12Z)-octadeca-9,12-dienyl)-1,3-dioxolan-4-yl)-N,N-dimethylethanamine in a molar ratio in the range of about 50-55;
(2) N-(chloromethyl)-2-(2,2-di((9Z,12Z)-octadeca-9,12-dienyl)-1,3-dioxolan-4-yl)-N,N-dimethylethanamonium chloride in a molar ratio in the range of about 2-5;
(3) 1,2-dipalmitoyl-sn-glycero-3-phosphocholine in a molar ratio in the range of about 6.9-7.2;
(4) cholesterol in a molar ratio in the range of about 33-34; and
(5) R-3-[(ω-methoxy-poly(ethyleneglycol)₂₀₀₀)carbamoyl]-1,2-dimyristyloxy-propyl-3-amine in a molar ratio in the range of about 5-6.

The present invention is also directed to any of the lipid nanoparticle compositions disclosed herein further comprising hyaluronan (or hyaluronic acid) as shown below:

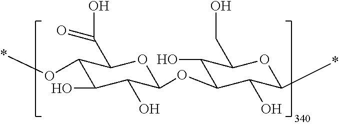

### General Syntheses of the Compounds of the Invention

Suitable processes for synthesizing compounds of formula I are provided in the examples. Generally, compounds of formula I can be prepared according to the schemes illustrated below. Unless otherwise indicated, the variables R, R¹, R², R³, and n are defined in the same manner as defined previously for the genus of formula I.

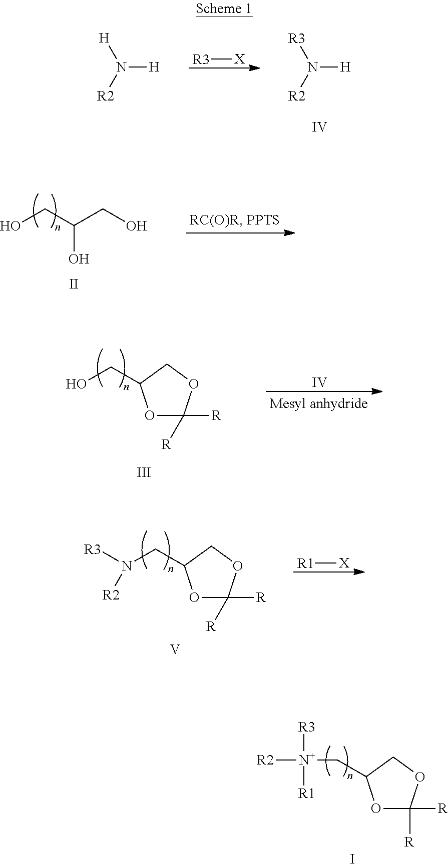

Compounds of interest (formula I) can be prepared according to Scheme 1. Starting with a mixture of an alkyl triol II and a appropriately substituted ketone, an acetal III is formed under dehydrating conditions. Coupling of compound III to either a commercially available or prepared secondary amine IV gives the tertiary amine V. A alkylation of compound V produces compounds of interest of interest I.

In the first step of this sequence, the intermediates III can be produced by reactions between the triol II and an appropriately substituted ketone. The reactions can be carried out in the presence of an acid such as pyridinium p-toluenesulfonate, in an inert solvent such as toluene, under dehydrating conditions such as in the presence of a Dean-Stark tube, at reflux temperature for several hours.

Coupling of primary alcohol III with secondary amine IV to give compound V can be easily accomplished using methods that are well known to someone skilled in the art. For example, primary alcohol III could first be transformed into the corresponding mesylate by adding methanesulfonyl anhydride in an anhydrous solvent such as methylene chloride in the presence of a base such as triethylamine. Compound V is then formed by mixing the resulting mesylate with an appropriately substituted secondary amine IV in an inert solvent such as tetrahydrofuran or dimethylformamide at a temperature between room temperature and reflux temperature for several hours.

Formation of the compound of interest I can be accomplished by alkylating tertiary amine V with an appropriately substituted alkyl halide or some similarly reactive, appropriately substituted alkylating reagent (for example alkyl mesylate alkyl tosylate, etc). In some instances, the alkyl halide may also serve as the solvent (for example methyl iodide) in other instances an inert solvent such as dimethylformamide might be necessary. These reactions typically are performed at a temperature between room temperature and reflux temperature for several hours and under an inert atmosphere such as nitrogen.

Suitable processes for synthesizing the lipid nanoparticles of the present invention are provided in Example 5.

### Utility

The compounds of formula I (such as those disclosed in examples 2-4) are useful in formulating lipid nanoparticle compositions to improve the delivery of nucleic acids such as siRNA to the cytoplasm of target cells to inhibit the expression of certain target proteins through RNA interference. Such lipid nanoparticle compositions are more efficacious and demonstrate improved knockdown capability compared to similar formulations lacking the compounds of formula I as shown in the examples.

Accordingly, the lipid nanoparticles of the present invention containing compounds of formula I may be used to encapsulate (or otherwise formulate) siRNAs inhibiting, for example, the expression of KIF11, KIF10, RRM2, FLT3, MLL, or RUNX1-RUNXT1. Cancer remains an important area of high unmet medical need. The majority of current treatments provide small gains in overall survival requiring a delicate balance between efficacy and toxicity. Cancer is characterized by uncontrolled growth and survival driven by the improper regulation of the cell cycle. The cell cycle is divided up into four stages culminating in cytokinesis. The cell cycle is designed to duplicate cellular material equally partitioning this material into what will become two new cells. Mitosis is the final stage and represents a highly regulated and coordinated process of moving the newly synthesized organelles, chromosomal DNA and other cell material into separate areas of the cell producing two new cells following cytokinesis. A critical step in mitosis is the proper positioning of chromosomal DNA at the center of the cell during metaphase. This ensures equal separation of DNA during the next step called anaphase. The movement and proper positioning of chromosomal DNA is accomplished by a family of motor proteins called kinesins. Motor proteins use the energy of ATP hydrolysis to move along microtubules and transport cellular cargo. The kinesins also play a key role in signaling the completion of movement of their cargo. KIF11 is the kinesin responsible for transporting chromosomal DNA to the metaphase plate. Thus, in particular embodiments the lipid nanoparticles of the present invention may be used to encapsulate (or otherwise formulate) siRNAs inhibiting the expression of KIF11 for the treatment of cancer. In other particular embodiments, the lipid nanoparticles of the present invention may be used to encapsulate (or otherwise formulate) siRNAs inhibiting the expression of KIF10, RRM2, FLT3, MLL, or RUNX1-RUNXT1 for the treatment of cancer or other diseases where the inhibition of such genes would be beneficial. In other embodiments, the lipid nanoparticles of the present invention may be used to encapsulate (or otherwise formulate) siRNAs inhibiting the expression of virtually any gene for the treatment of any disease or disorder where the inhibition of such a gene(s) would be beneficial.

The lipid nanoparticles of the present invention can be administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations as the minimum amount necessary to inhibit the expression of the target protein and avoid unacceptable toxicity. For example, such amount may be below the amount that is toxic to normal cells, or the mammal as a whole.

The compounds of the invention may be administered by parenteral, intraperitoneal, intrapulmonary, or by other known forms of administration. Parenteral infusions include intramuscular, intravenous, intraarterial, intraperitoneal, or subcutaneous administration.

### EXAMPLES

The invention will be more fully understood by reference to the following examples. They should not, however, be construed as limiting the scope of the invention. Abbreviations used herein are as follows: CAD=corona charged aerosol detector, DCM=dichloromethane, HPLC=high pressure liquid chromatography, MeOH=methanol, and MS=mass spectrometry.

### Example 1

### Synthesis of 2-(2,2-di((9Z,12Z)-octadeca-9,12-dienyl)-1,3-dioxolan-4-yl)-N,N-dimethylethanamine:

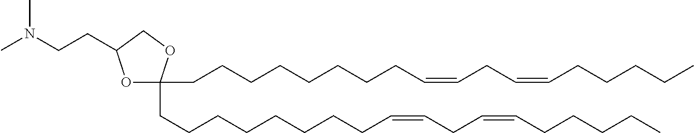 [SMILES: CCCCC/C=C\C/C=C\CCCCCCCCC1(CCCCCCCC/C=C\C/C=C\CCCCC)OCC(CCN(C)C)O1]

This lipid is known and can be prepared using methods published in International Publication No. WO 2010/088537 (Int. App. No. PCT/US2010/022614) by Akinc et al.; International Publication No. WO 2010/048536 (Int. App. No. PCT/US2009/061897) by Manoharan et al.; International Publication No. WO 2010/042877 (Int. App. No. PCT/US2009/060251) by Hope et al.; and/or Semple, SC et al. Nature Biotechnology, 28, 172-176 (2010), all of which are hereby incorporated by reference in their entirety; or by using alternative methods known in the art.

### Example 2

### Synthesis of 2-(2,2-di((9Z,12Z)-octadeca-9,12-dienyl)-1,3-dioxolan-4-yl)-N-ethyl-N,N-dimethylethanamonium iodide:

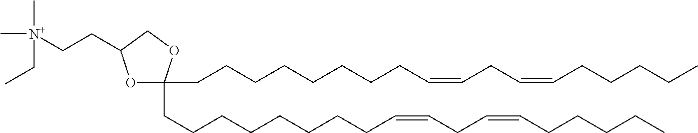 [SMILES: CCCCC/C=C\C/C=C\CCCCCCCCC1(CCCCCCCC/C=C\C/C=C\CCCCC)OCC(CC[N+](C)(C)CC)O1]

In accordance with scheme 1 disclosed previously, 2-(2,2-di((9Z,12Z)-octadeca-9,12-dienyl)-1,3-dioxolan-4-yl)-N,N-dimethylethanamine (2.6 g) was dissolved in heptane (2.6 mL), the solution was cooled to 0° C., and added ethyl iodide (3.4 mL). The reaction was allowed to warm up to ambient temperature, then heated to 40° C. for 16 h. TLC (DCM-MeOH 9:1) showed complete conversion. The solution was concentrated under vacuum at 25° C., then redissolved in heptane and concentrated 3× with heptane to remove volatile byproducts to obtain 2-(2,2-di((9Z,12Z)-octadeca-9,12-dienyl)-1,3-dioxolan-4-yl)-N-ethyl-N,N-dimethylethanamonium iodide, 3.2 g. HPLC 89.61% (CAD detector); MS 670.7.

In accordance with scheme 1 disclosed previously, 2-(2,2-di((9Z,12Z)-octadeca-9,12-dienyl)-1,3-dioxolan-4-yl)-N,N-dimethylethanamine (2.6 g) was dissolved in heptane (2.6 mL), the solution was cooled to 0° C., and added ethyl iodide (3.4 mL). The reaction was allowed to warm up to ambient temperature, then heated to 40° C. for 16 h. TLC (DCM-MeOH 9:1) showed complete conversion. The solution was concentrated under vacuum at 25° C., then redissolved in heptane and concentrated 3× with heptane to remove volatile byproducts to obtain 2-(2,2-di((9Z,12Z)-octadeca-9,12-dienyl)-1,3-dioxolan-4-yl)-N-ethyl-N,N-dimethylethanamonium iodide, 3.2 g. HPLC 89.61% (CAD detector); MS 670.7 .Example 3. Synthesis of 2-(2,2-di((9Z,12Z)-octadeca-9,12-dienyl)-1,3-dioxolan-4-yl)-N,N,N-trimethylethanamonium iodide:

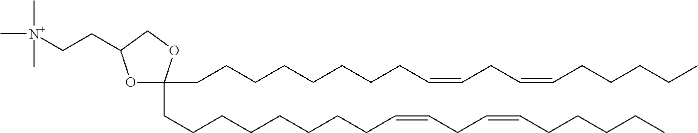 [SMILES: CCCCC/C=C\C/C=C\CCCCCCCCC1(CCCCCCCC/C=C\C/C=C\CCCCC)OCC(CC[N+](C)(C)C)O1]

In accordance with scheme 1 disclosed previously, 2-(2,2-di((9Z,12Z)-octadeca-9,12-dienyl)-1,3-dioxolan-4-yl)-N,N-dimethylethanamine (2.4 g) was dissolved in heptane (25 mL), the solution was cooled to 0° C., and added methyl iodide (2.4 mL). The reaction was allowed to warm up to ambient temperature and stirred for 6 h. TLC (DCM-MeOH 9:1) indicated complete conversion. The solution was concentrated under vacuum at 25° C., then redissolved in heptane and concentrated 3× with heptane to remove volatile byproducts to obtain 2-(2,2-di((9Z,12Z)-octadeca-9,12-dienyl)-1,3-dioxolan-4-yl)-N,N,N-trimethylethanamonium iodide, 3.0 g. HPLC 96.44% (CAD detector); MS 656.7.

### Example 4

### Synthesis of N-(chloromethyl)-2-(2,2-di((9Z,12Z)-octadeca-9,12-dienyl)-1,3-dioxolan-4-yl)-N,N-dimethylethanamonium chloride

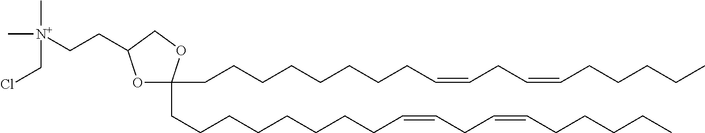 [SMILES: CCCCC/C=C\C/C=C\CCCCCCCCC1(CCCCCCCC/C=C\C/C=C\CCCCC)OCC(CC[N+](C)(C)CCl)O1]

In accordance with scheme 1 disclosed previously, 2-(2,2-di((9Z,12Z)-octadeca-9,12-dienyl)-1,3-dioxolan-4-yl)-N,N-dimethylethanamine (3 g) was dissolved in dichloromethane (50 mL). The clear solution was heated to 40° C. for 24 h, and then continued stirring at ambient under nitrogen for 7 days. The reaction was concentrated under vacuum and the solvent was exchanged to heptane and purified on 50 g of flash silica gel pretreated with heptane. The heptane solution (40 mL) was applied directly to the chromatography column containing 50 g of silica gel pretreated with heptane and further eluted with heptane. A total 70 mL of eluate were collected and concentrated to obtain 1.7 g of N-(chloromethyl)-2-(2,2-di((9Z,12Z)-octadeca-9,12-dienyl)-1,3-dioxolan-4-yl)-N,N-dimethylethanamonium chloride as a colorless gum. HPLC 96.38% (CAD detector); MS 690.

### Example 5

### Formation of Lipid Nanoparticles

Lipid nanoparticles comprising: (1) the compound of example 1: 2-(2,2-di((9Z,12Z)-octadeca-9,12-dienyl)-1,3-dioxolan-4-yl)-N,N-dimethyl-ethanamine; (2) a compound of formula I, (3) phospholipids, (4) cholesterol, (5) pegylated lipids, and (6) siRNA; and variations thereof were formed using an ethanol injection method. Specifically,

Table 1 (below) provides a description of the specific ingredients and molar ratios for each lipid nanoparticle formulation that was synthesized. Table 2 provides a description of the diameter, the percent of siRNA that was encapsulated (if measured), and the zeta potential (if measured) of each lipid nanoparticle formulation that was synthesized. The lipid portion of each formulation [(1)-(5) above] was dissolved in absolute ethanol (200 proof). In a separate vial (borosilicate, Schott Fiolax Clear), the siRNA portion (6) was diluted to an appropriate concentration (0.05-2 mg/mL) using either nuclease-free water (Qiagen), citrate buffer (20 mM, pH 4.0), or 5% Dextrose (D5W). The lipid-ethanol solution was then added with rapid stirring to the aqueous siRNA containing solution resulting in a final ethanol concentration of 10-50%. After standing at room temperature for 3-24 hours, under argon and shielded from the light, the formed lipid nanoparticle suspension was transferred to 10,000 MWCO dialysis cassettes (Slide-A-Lyzer, Thermo Scientific) and dialyzed against either phosphate buffered saline or 5% Dextrose (D5W) for 12-24 hours at room temperature. The lipid nanoparticles were filtered using a 0.22 micron syringe filter (Millex-GV, Millipore) prior to use. The following are definitions for the abbreviations used in Table 1 for each formulation number (Form #):

For the compound of Example 1:
Dlin-KC2-DMA=2-(2,2-di((9Z,12Z)-octadeca-9,12-dienyl)-1,3-dioxolan-4-yl)-N,N-dimethylethanamine;

For compounds of formula I:
DLin-KC2-C1MDMA =N-(chloromethyl)-2-(2,2-di((9Z,12Z)-octadeca-9,12-dienyl)-1,3-dioxolan-4-yl)-N,N-dimethylethanamonium chloride;
DLin-KC2-TMA=2-(2,2-di((9Z,12Z)-octadeca-9,12-dienyl)-1,3-dioxolan-4-yl)-N,N,N-trimethylethanamonium iodide;
DLin-KC2-DMEA=2-(2,2-di((9Z,12Z)-octadeca-9,12-dienyl)-1,3 -dioxolan-4-yl)-N-ethyl-N,N-dimethylethanamonium iodide;

For the phospholipids:
DPPC 32 1,2-dipalmitoyl-sn-glycero-3-phosphocholine;
DSPC=1,2-d istearoyl-sn-glycero-3 -phosphocholine;
DOPC=1,2-dioleoyl-sn-glycero-3-phosphocholine;

Cholesterol=cholesterol

For the pegylated lipids:

PEG-c-DOMG =R-3-[(ω-methoxy-poly(ethyleneglycol)₂₀₀₀)carbamoyl]-1,2-dimyristyloxy-propyl-3-amine.

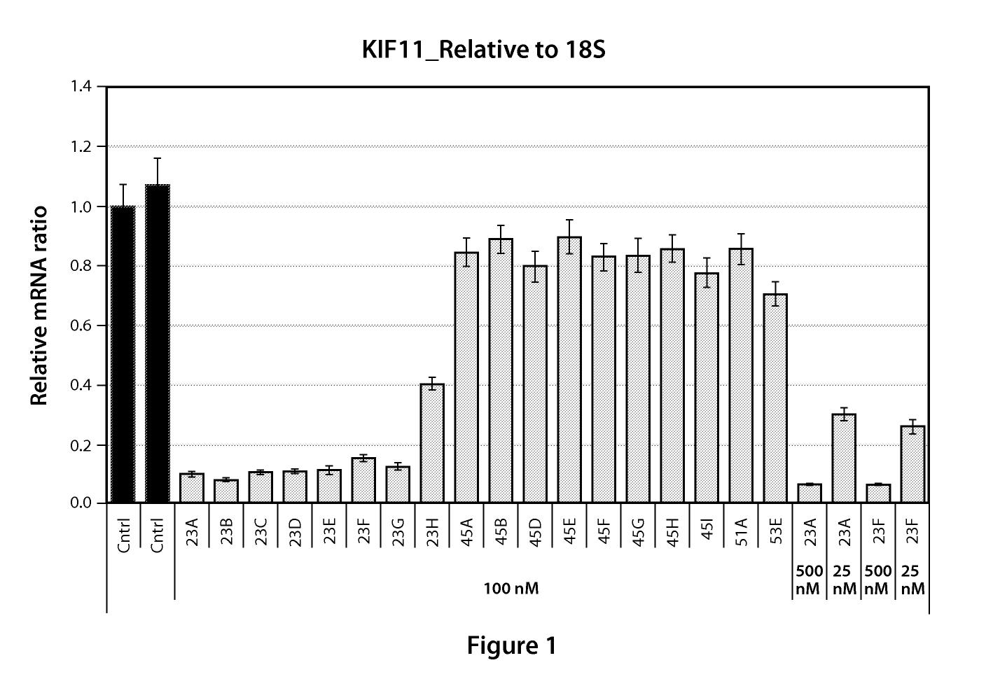

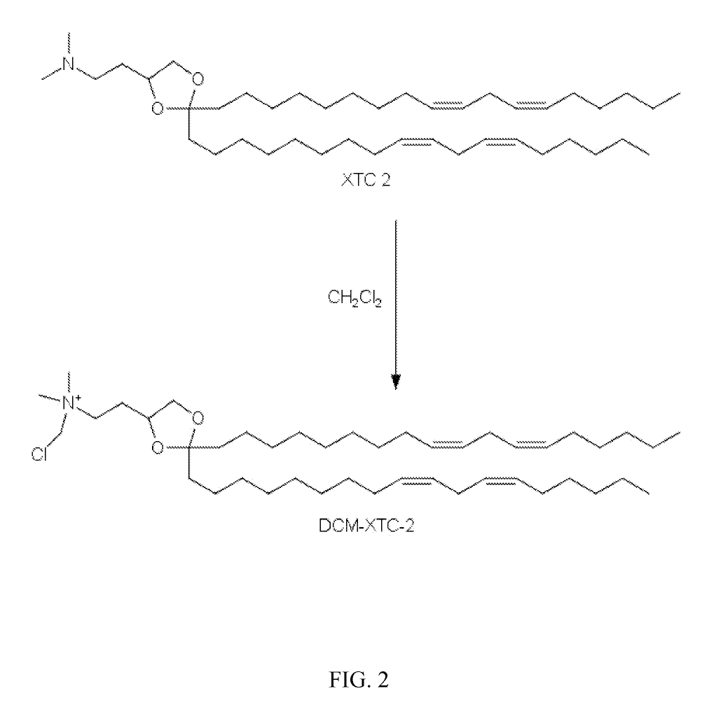

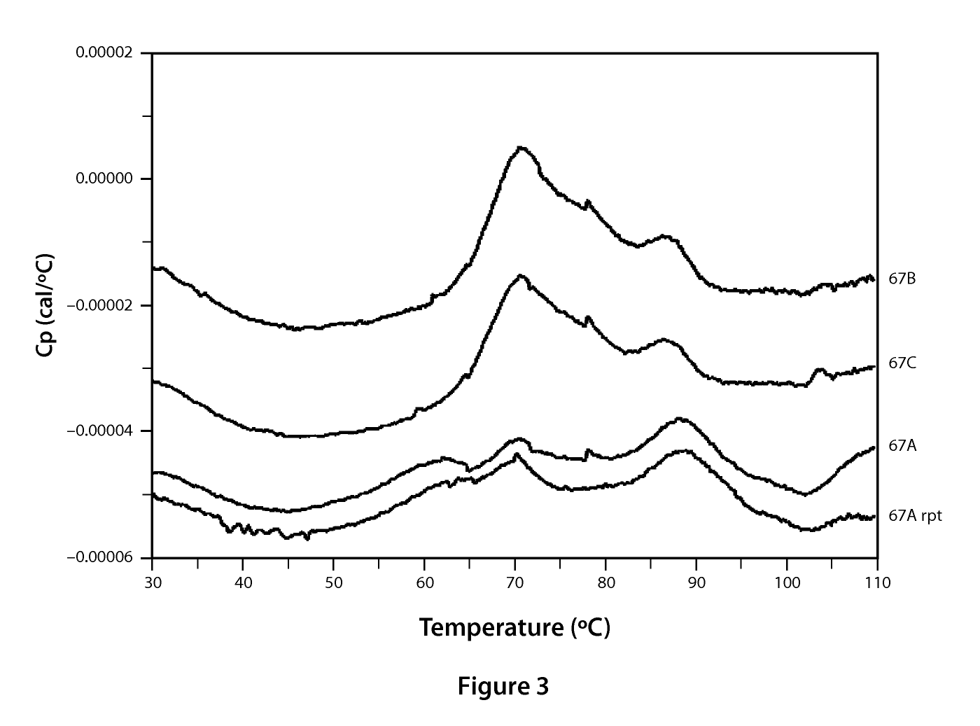

For example, in Table 1 the composition for formulation 23A (Dlin-KC2-DMA/DLin-KC2-CIMDMA/DPPC/Cholesterol/PEG-c-DOMG) indicates that this lipid nanoparticle is comprised of:
(1) 2-(2,2-di((9Z,12Z)-octadeca-9,12-dienyl)-1,3-dioxolan-4-yl)-N,N-dimethylethanamine, with a molar ratio of 52,
(2) N-(chloromethyl)-2-(2,2-di((9Z,12Z)-octadeca-9,12-dienyl)-1,3-dioxolan-4-yl)-N,N-dimethylethanamonium chloride, with a molar ratio of 2.1,
(3) 1,2-dipalmitoyl-sn-glycero-3-phosphocholine, with a molar ratio of 6.9,
(4) cholesterol, with a molar ratio of 33.2, and
(5) R-3-[(ω-methoxy-poly(ethyleneglycol)₂₀₀₀)carbamoyl]-1,2-dimyristyloxy-propyl-3 -amine, with a molar ratio of 5.8,
   and has an N/P ratio of 1.92, and (as shown in Table 2) has a particle diameter of 106.2 nm, a siRNA encapsulation of 72.4%, with results shown in FIG. 1. The N/P ratio is the molar ratio of cationic lipid amino groups to siRNA phosphate groups. The siRNA used in these formulations was 21 nucleotides in length with 2 nucleotide overhangs which targeted human KIF11 or Firefly Luciferase mRNA. The KiF11 sequence is comprised of:
ucGAGAAucuAAAcuAAcudTsdT 5′→3′ sense (SEQ ID 1) and
AGUuAGUUuAGAUUCUCGAdTsdT 5′→3′ antisense strands (SEQ ID 2),
   where lower case lettering=2′-OMe RNA modification, upper case lettering=normal RNA, dT=deoxythymidine, and s=phosphorothioate. The Luciferase sequence is comprised of
cuuAcGcuGAGuAcuucGAdTsdT 3′ sense (SEQ ID 3) and
UCGAAGuACUcAGCGuAAGdTsdT 5′→3′ antisense strands (SEQ ID 4).

TABLE 1
Lipid nanoparticle compositions.

| Form. | Lipid Composition | Molar Ratio |
|---|---|---|
| 23A | Dlin-KC2-DMA/DLin-KC2-CIMDMA/DPPC/Cholesterol/PEG-c-DOMG | 52/2.1/6.9/33.2/5.8 |
| 23B | Dlin-KC2-DMA/DLin-KC2-CIMDMA/DPPC/Cholesterol/PEG-c-DOMG | 52.4/2.1/6.9/33.4/5.1 |
| 23C | Dlin-KC2-DMA/DLin-KC2-CIMDMA/DPPC/Cholesterol/PEG-c-DOMG | 52.8/2.1/7.0/33.7/4.4 |
| 23D | Dlin-KC2-DMA/DLin-KC2-CIMDMA/DPPC/Cholesterol/PEG-c-DOMG | 53.2/2.1/7.0/34.0/3.7 |
| 23E | Dlin-KC2-DMA/DLin-KC2-CIMDMA/DPPC/Cholesterol/PEG-c-DOMG | 53.6/2.1/7.1/34.2/3.0 |
| 23F | Dlin-KC2-DMA/DLin-KC2-CIMDMA/DPPC/Cholesterol/PEG-c-DOMG | 54.0/2.2/7.1/34.4/2.3 |
| 23G | Dlin-KC2-DMA/DLin-KC2-CIMDMA/DPPC/Cholesterol/PEG-c-DOMG | 54.4/2.2/7.2/34.7/1.5 |
| 23H | Dlin-KC2-DMA/DLin-KC2-CIMDMA/DPPC/Cholesterol/PEG-c-DOMG | 54.9/2.2/7.2/34.9/0.8 |
| 45A | Dlin-KC2-DMA/DPPC/Cholesterol/PEG-c-DOMG | 56.7/7.2/34.7/1.5 |
| 45B | Dlin-KC2-DMA/DPPC/Cholesterol/PEG-c-DOMG | 56.7/7.2/34.7/1.5 |
| 45C | Dlin-KC2-DMA/DPPC/Cholesterol/PEG-c-DOMG | 56.7/7.2/34.7/1.5 |
| 45D | Dlin-KC2-DMA/DPPC/Cholesterol/PEG-c-DOMG | 56.7/7.2/34.7/1.5 |
| 45E | Dlin-KC2-DMA/DPPC/Cholesterol/PEG-c-DOMG | 56.7/7.2/34.7/1.5 |
| 45F | Dlin-KC2-DMA/DPPC/Cholesterol/PEG-c-DOMG | 56.7/7.2/34.7/1.5 |
| 45G | Dlin-KC2-DMA/DPPC/Cholesterol/PEG-c-DOMG | 56.7/7.2/34.7/1.5 |
| 45H | Dlin-KC2-DMA/DPPC/Cholesterol/PEG-c-DOMG | 56.7/7.2/34.7/1.5 |
| 45I | Dlin-KC2-DMA/DPPC/Cholesterol/PEG-c-DOMG | 56.7/7.2/34.7/1.5 |
| 51A | Dlin-KC2-DMA/DPPC/Cholesterol/PEG-c-DOMG | 54.1/6.8/33.2/5.8 |
| 53E | Dlin-KC2-DMA/DPPC/Cholesterol/PEG-c-DOMG | 56.7/7.2/34.7/1.5 |
| 67A | Dlin-KC2-DMA/DLin-KC2-CIMDMA/DPPC/Cholesterol/PEG-c-DOMG | 52.7/1.5/6.9/33.2/5.8 |
| 67B | Dlin-KC2-DMA/DPPC/Cholesterol/PEG-c-DOMG | 54.1/6.9/33.2/5.8 |
| 67C | Dlin-KC2-DMA/DPPC/Cholesterol/PEG-c-DOMG | 54.1/6.9/33.2/5.8 |
| 85A | Dlin-KC2-DMA/DPPC/Cholesterol/PEG-c-DOMG | 54.1/6.9/33.2/5.8 |
| 85B | Dlin-KC2-DMA/DLin-KC2-CIMDMA/DPPC/Cholesterol/PEG-c-DOMG | 19.9/34.2/6.9/33.2/5.8 |
| 85C | Dlin-KC2-DMA/DLin-KC2-CIMDMA/DPPC/Cholesterol/PEG-c-DOMG | 38.2/15.9/6.9/33.2/5.8 |
| 85D | Dlin-KC2-DMA/DLin-KC2-CIMDMA/DPPC/Cholesterol/PEG-c-DOMG | 51.9/2.2/6.9/33.2/5.8 |
| 85E | Dlin-KC2-DMA/DLin-KC2-CIMDMA/DPPC/Cholesterol/PEG-c-DOMG | 49.7/4.4/6.9/33.2/5.8 |
| 85F | Dlin-KC2-DMA/DLin-KC2-CIMDMA/DPPC/Cholesterol/PEG-c-DOMG | 45.3/8.8/6.9/33.2/5.8 |
| 85G | Dlin-K-DMA/DPPC/Cholesterol/PEG-c-DOMG | 54.1/6.9/33.2/5.8 |
| 85H | Dlin-KC2-DMA/DLin-KC2-CIMDMA/DPPC/Cholesterol/PEG-c-DOMG | 52.7/1.5/6.9/33.2/5.8 |
| 91D | Dlin-KC2-DMA/DLin-KC2-CIMDMA/DPPC/Cholesterol/PEG-c-DOMG | 51.9/2.2/6.9/33.2/5.8 |
| 91E | Dlin-KC2-DMA/DLin-KC2-CIMDMA/DPPC/Cholesterol/PEG-c-DOMG | 51.9/2.2/6.9/33.2/5.8 |
| 91G | Dlin-KC2-DMA/DLin-KC2-CIMDMA/DPPC/Cholesterol/PEG-c-DOMG | 51.9/2.2/6.9/33.2/5.8 |
| 91I | Dlin-KC2-DMA/DLin-KC2-CIMDMA/DPPC/Cholesterol/PEG-c-DOMG | 51.9/2.2/6.9/33.2/5.8 |
| 95A | Dlin-KC2-DMA/DLin-KC2-CIMDMA/DPPC/Cholesterol/PEG-c-DOMG | 49.7/4.4/6.9/33.2/5.8 |
| 95B | Dlin-KC2-DMA/DLin-KC2-CIMDMA/DPPC/Cholesterol/PEG-c-DOMG | 49.7/4.4/6.9/33.2/5.8 |
| 95C | Dlin-KC2-DMA/DLin-KC2-CIMDMA/DPPC/Cholesterol/PEG-c-DOMG | 49.7/4.4/6.9/33.2/5.8 |
| 95D | Dlin-KC2-DMA/DLin-KC2-CIMDMA/DPPC/Cholesterol/PEG-c-DOMG | 49.7/4.4/6.9/33.2/5.8 |
| 95E | Dlin-KC2-DMA/DLin-KC2-CIMDMA/DPPC/Cholesterol/PEG-c-DOMG | 51.9/2.2/6.9/33.2/5.8 |
| 95F | Dlin-KC2-DMA/DLin-KC2-CIMDMA/DPPC/Cholesterol/PEG-c-DOMG | 51.9/2.2/6.9/33.2/5.8 |
| 95G | Dlin-KC2-DMA/DLin-KC2-CIMDMA/DPPC/Cholesterol/PEG-c-DOMG | 51.9/2.2/6.9/33.2/5.8 |
| 95H | Dlin-KC2-DMA/DLin-KC2-CIMDMA/DPPC/Cholesterol/PEG-c-DOMG | 49.7/4.4/6.9/33.2/5.8 |
| 95I | Dlin-KC2-DMA/DLin-KC2-CIMDMA/DPPC/Cholesterol/PEG-c-DOMG | 51.9/2.2/6.9/33.2/5.8 |
| 123A | Dlin-KC2-DMA/DPPC/Cholesterol/PEG-c-DOMG | 54.1:6.9:33.2:5.8 |
| 123B | DLin-KC2-TMA/DPPC/Cholesterol/PEG-c-DOMG | 54.1:6.9:33.2:5.8 |
| 123C | DLin-KC2-DMEA/DPPC/Cholesterol/PEG-c-DOMG | 54.1:6.9:33.2:5.8 |
| 123D | Dlin-KC2-DMA/DLin-KC2-TMA/DPPC/Cholesterol/PEG-c-DOMG | 49.8:4.40:6.9:33.2:5.8 |
| 123E | Dlin-KC2-DMA/DLin-KC2-DMEA/DPPC/Cholesterol/PEG-c-DOMG | 49.8:4.40:6.9:33.2:5.8 |
| 123F | Dlin-KC2-DMA/DLin-KC2-CIMDMA/DPPC/Cholesterol/PEG-c-DOMG | 49.8:4.40:6.9:33.2:5.8 |
| 123G | Dlin-KC2-DMA/DLin-KC2-TMA/DPPC/Cholesterol/PEG-c-DOMG | 45.4:8.8:6.9:33.2:5.8 |
| 123H | Dlin-KC2-DMA/DLin-KC2-DMEA/DPPC/Cholesterol/PEG-c-DOMG | 45.4:8.8:6.9:33.2:5.8 |
| 123I | Dlin-KC2-DMA/DLin-KC2-CIMDMA/DPPC/Cholesterol/PEG-c-DOMG | 45.4:8.8:6.9:33.2:5.8 |

TABLE 2
Lipid nanoparticle characterization.

| | siRNA/ | | | Particle | | |
|---|---|---|---|---|---|---|
| | HA Wt. | N/P | Results shown | Diameter | % siRNA | Zeta Potential |
| Form. # | Ratio | Ratio | in FIG. | (nm) | Encapsulated | (MeV) |
| 23A | N/A | 1.92 | 1 | 106.2 | 72.4 | Not Measured |
| 23B | N/A | 1.92 | 1, 4, 7 | 122.7 | 76.1 | Not Measured |
| 23C | N/A | 1.92 | 1, 5 | 122.7 | 87.5 | Not Measured |
| 23D | N/A | 1.92 | 1 | 132.4 | 86.6 | Not Measured |
| 23E | N/A | 1.92 | 1 | 132.8 | 80 | Not Measured |
| 23F | N/A | 1.92 | 1 | 148.3 | 78.3 | Not Measured |
| 23G | N/A | 1.92 | 1 | 186.6 | 59 | Not Measured |
| 23H | N/A | 1.92 | 1 | 252 | 75 | Not Measured |
| 45A | N/A | 1.44 | 1 | 179.5 | Not Measured | Not Measured |
| 45B | N/A | 1.92 | 1 | 197.5 | Not Measured | Not Measured |
| 45C | N/A | 2.4 | | 203.6 | Not Measured | Not Measured |
| 45D | N/A | 2.88 | 1 | 207.1 | Not Measured | Not Measured |
| 45E | N/A | 3.36 | 1 | 207.9 | Not Measured | Not Measured |
| 45F | N/A | 3.84 | 1 | 168.3 | Not Measured | Not Measured |
| 45G | N/A | 2.4 | 1 | 209.6 | Not Measured | Not Measured |
| 45H | N/A | 2.88 | 1 | 215.2 | Not Measured | Not Measured |
| 45I | N/A | 3.84 | 1 | 218.2 | Not Measured | Not Measured |
| 51A | N/A | 1.92 | 1 | 156.2 | 66.9 | −3.38 |
| 53E | N/A | 3.84 | 1 | 204.9 | 81.6 | −0.05 |
| 67A | N/A | 1.92 | 3, 4, 8 | 152.9 | 85.6 | −3.47 |
| 67B | N/A | 1.92 | 3, 8 | 170.5 | 77.6 | −3.36 |
| 67C | N/A | 1.92 | 3 | 158.2 | 68.2 | −4.43 |
| 85A | N/A | 1.92 | 4 | 178.7 | 94.5 | Not Measured |
| 85B | N/A | 1.92 | 4 | 193 | 94.4 | Not Measured |
| 85C | N/A | 1.92 | 4 | 155.4 | 85.5 | Not Measured |
| 85D | N/A | 1.92 | 4 | 179.2 | 90.5 | Not Measured |
| 85E | N/A | 1.92 | 4 | 170.7 | 94.3 | Not Measured |
| 85F | N/A | 1.92 | 4 | 159.9 | 87.3 | Not Measured |
| 85G | N/A | 1.92 | 4 | 185.6 | 89.3 | Not Measured |
| 85H | N/A | 1.92 | 4 | 167.9 | 93.8 | Not Measured |
| 91D | 0 | 3.36 | 10 | 149.2 | 77.1 | −1.26 |
| 91E | 0.25 | 3.84 | | 164.8 | 94.7 | 0.061 |
| 91G | 0.5 | 3.36 | 11 | 199.4 | 90.1 | 1.88 |
| 91I | 0 | 3.36 | 9 | 151.1 | 90.3 | −0.279 |
| 95A | N/A | 2.31 | 7 | 175.3 | 87.1 | Not Measured |
| 95B | N/A | 3.46 | 7 | 157.8 | 94.2 | Not Measured |
| 95C | N/A | 4.62 | 7 | 155.7 | 74.1 | Not Measured |
| 95D | N/A | 5.77 | 7 | 155.3 | 93.0 | Not Measured |
| 95E | N/A | 4.04 | 7 | 161 | 80.5 | Not Measured |
| 95F | N/A | 6.06 | 7 | 167.2 | 97.2 | Not Measured |
| 95G | N/A | 8.08 | 7 | 157.9 | 80.8 | Not Measured |
| 95H | 1 | 5.77 | 7 | 177 | 90.0 | Not Measured |
| 95I | 1 | 8.08 | 7 | 161 | 80.6 | Not Measured |
| 123A | N/A | 1.92 | 5 | 142.2 | 72.7 | −0.42 |
| 123B | N/A | 1.92 | 5 | 98.9 | 91.2 | −4.04 |
| 123C | N/A | 1.92 | 5 | 110 | 90.4 | −1.51 |
| 123D | N/A | 1.92 | 5 | 152.2 | 86.9 | −1.15 |
| 123E | N/A | 1.92 | 5 | 148.8 | 75 | −2.69 |
| 123F | N/A | 1.92 | 5 | 138.9 | 85.5 | −2.08 |
| 123G | N/A | 1.92 | 5 | 150.8 | 85.2 | −1.28 |
| 123H | N/A | 1.92 | 5 | 156.8 | 85.8 | −1.67 |
| 123I | N/A | 1.92 | 5 | 139.4 | 87.8 | −1.81 |

siRNA Concentration and % Encapsulation. The ribogreen fluorescence assay was used to determine the concentration and percent entrapment of siRNA in each formulation preparation. Ribogreen dye fluoresces upon binding with double stranded nucleic acids and can thus be used to measure the amount of free siRNA in a sample. The amount of bound/encapsulated siRNA can be calculated after determining both the free/unbound siRNA and total siRNA concentrations. The total amount of siRNA can be obtained after disrupting the lipid nanoparticle and thus liberating the siRNA by using a surfactant such as Triton X100. For this body of work, a commercially available ribogreen assay kit (Quanti-iT™ Ribogreen® RNA Assay Kit by Invitrogen) was used per manufacturer's instructions. siRNA sequences used in the preparation of the lipid nanoparticle (LNP) were used as standards. 1% Triton ×100 was used to disrupt the lipid nanoparticle suspension for the purpose of determining the total siRNA content. Ribogreen fluorescence was measured with a SpectraMax M5 (Molecular Devices) multi-mode microplate reader using excitation and emission wavelengths of 485 and 525 nm, respectively. The percent encapsulated siRNA was determined by using the following equation: % Encapsulation=([siRNA]total−[siRNA]free)/[siRNA]total×100. [siRNA]total=the total concentration (mg/ml) of siRNA measured after disruption of the lipid nanoparticle. [siRNA]=the concentration (mg/ml) of free/unbound siRNA measured prior to the addition of Triton X-100. For all in vitro screening, the concentration of bound/encapsulated siRNA was used for dosing. For all in vivo experiments, the total siRNA concentration was used to determine dosing. The results (% siRNA encapsulated) for each formulation (if measured) are shown in Table 2.

Particle Size and Zeta Potential. Particle size and zeta potential were determined using a Malvern Zetasizer Nano-ZS Instrument. For particle size measurements, the lipid nanoparticles were diluted by a factor of 1 to 100 (204 diluted in 2000 μL buffer) using either phosphate buffered saline (PBS), 5% dextrose (D5W), or 20 mM citrate buffer (pH 4.0). Light scattering measurements were performed at 25° C. in polystyrene cuvettes. For zeta potential measurements, the lipid nanoparticles were diluted using nuclease free water using a dilution factor of 1 to 100 (20 μL diluted in 2000 μL DI water). Both the particle size and zeta potential appeared to be somewhat dependent on the specific lipid composition and relative amount of lipid to siRNA used to generate the particle. The particle size and zeta potential for each formulation (if measured) are shown in Table 2.

In Vitro Evaluation of Formulated Lipid Nanoparticle Activity

Cell Lines: The human cancer cell lines PC3, MV4-11, K562, KG-1, HEL, THP-1 (ATCC, Manassas, Va.) and MOLM13 (DSMZ, Braunschweig, Germany) were maintained in media (DMEM for PC3 and RMPI for the other cell lines) which was supplemented with 10% heat-inactivated Fetal Bovine Serum (HI-FBS; Gibco/BRL, Gaithersburg, Md.) and 2 mM L-glutamine (Gibco/BRL).

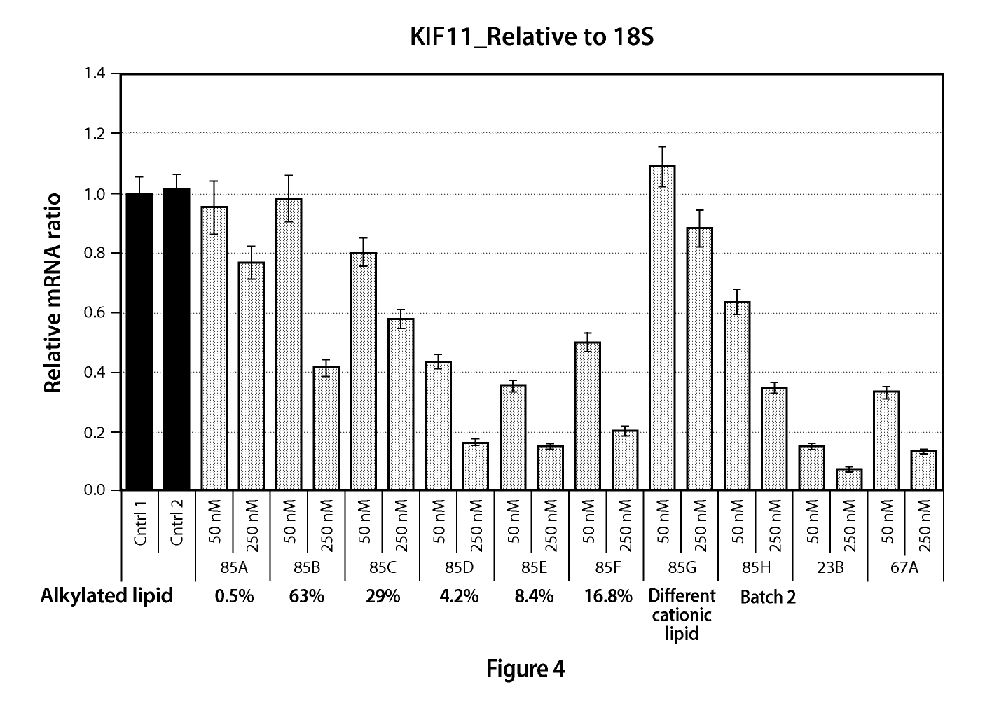

Transfection: 2×10⁴ PC3 or 2×10⁵ suspension cells in 1 ml of culturing medium were seeded in 24-well plates 24 hours before transfection and RNA quantification targeting siRNA were formulated in LNPs and directly added to the medium for transfection at indicated siRNA concentrations shown in FIGS. 1, 4, 5(a,b), 7, and 8. Cells were then collected for RNA quantification.

Sample collection and mRNA purification for in vitro studies were performed as follows. PC3 cells were lysed directly on the plate with RNA lysis buffer (Qiagen). Suspension cells were collected into tubes and spun down at 2,000 rpm for 1 min, then the cell pellets were lysed with RNA lysis buffer (Qiagen). Total RNA from all collected samples was purified using Qiagen RNeasy Kit following the manufacturer's protocol. Relative quantification of target mRNA and 18S ribosomal RNA gene expression was carried out with cDNA Reverse Transcription Reagents from Applied Biosystems followed by Taqman Gene Expression Assays (Applied Biosystems) using the manufacturer's protocol. The catalog numbers for each probe set were: human KIF 11 (Hs00946303_ml) and 18S (4319413E). The results are shown FIGS. 1, 4, 5(a,b), 7, and 8.

Comparative microarray: The indicated cell lines shown in table 3 were grown in tissue culture following the manufacturers recommended conditions. Total RNA was isolated from cells using the Qiagen RNeasy Mini Kit (QIAGEN, Valencia, Calif.) and quality was assessed on the Agilent Bioanalyzer 2100 (Santa Clara, Calif.). 15 μg of total RNA was converted into cDNA and cRNA according to the manufacturer's recommendation and using manufacturer's kits (One-cycle cDNA Synthesis Kit with the addition of reagents from the Poly-A RNA Control Kit, IVT Labeling Kit, Sample Cleanup Module, Control Kit and Control Oligo B2, Affymetrix, Santa Clara, Calif.). Hybridization Mix was hybridized first to Affymetrix Human Genome U133 plus 2.0 Arrays. Staining and washing steps were performed as suggested by the manufacturer (Affymetrix, Santa Clara, Calif.). Each hybridized Affymetrix GeneChip array was scanned with a GeneChip Scanner 3000 7G (Agilent/Affymetrix). Image analysis was done with the Affymetrix GCOS software.

For the statistical analysis of the expression measurements, an in-house implementation of the RMA algorithm was used to perform the background correction, normalization and signal summarization. A multifactor ANOVA model with linear contrasts was applied to identify differentially expressed genes as a result of compound treatments and time effects using the Partek Genomic Suite (Partek Inc., St. Louis, Mo.).

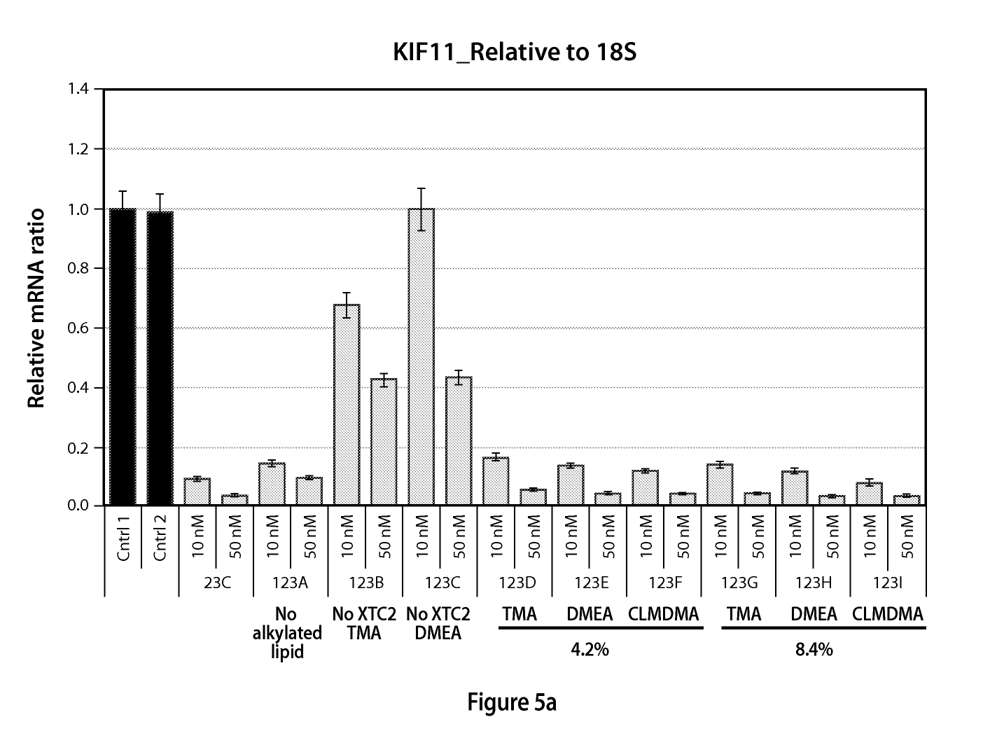

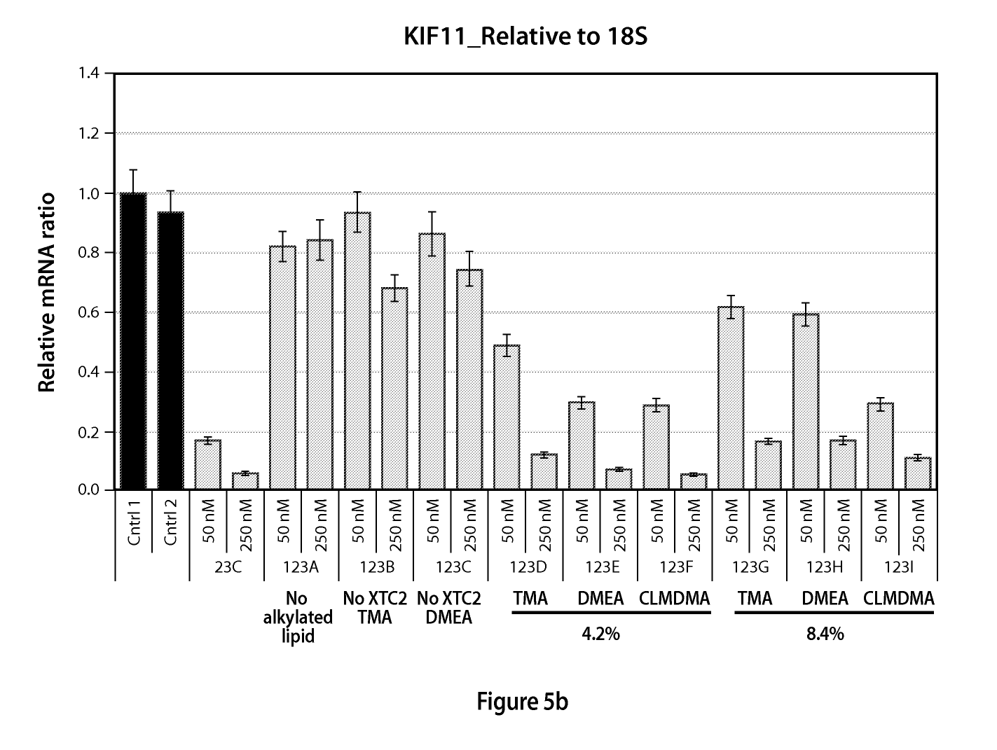

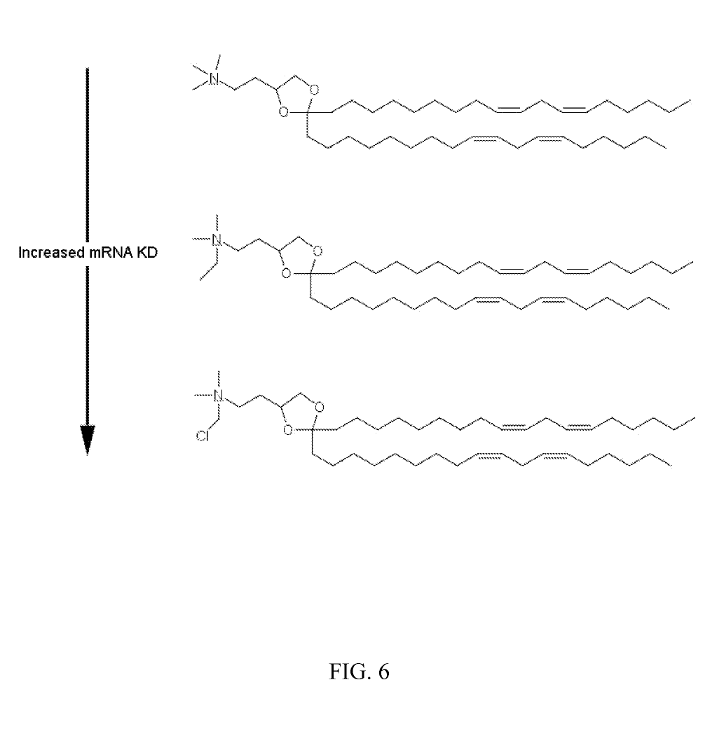

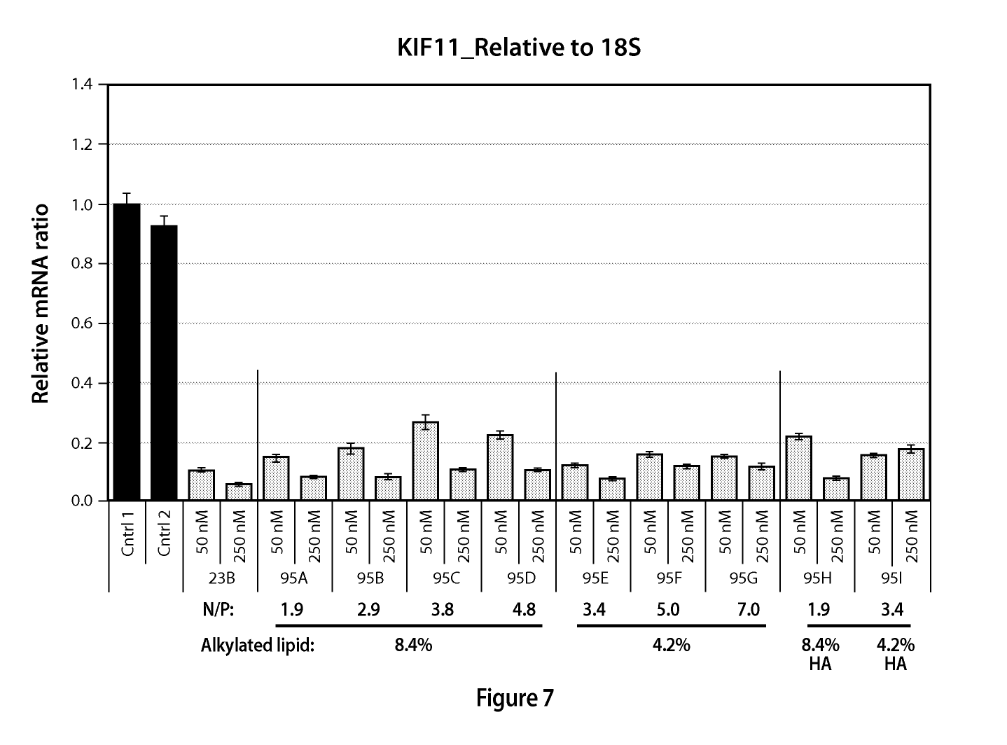

Generally, ethanolic solutions containing the compound of example 1: 2-(2,2-di((9Z,12Z)-octadeca-9,12-dienyl)-1,3-dioxolan-4-yl)-N,N-dimethylethanamine, the facilitating phosphor-lipid of 1,2-dipalmitoyl-sn-glycero-3-phosphocholine, cholesterol, and a pegylated lipid at differing relative amounts (see Table 1) were mixed in an aqueous solution containing siRNA. The ethanol was then removed using dialysis. The experiments indicate that these solutions were effective in delivering siRNA to adherence cell cultures such as PC3 cells (prostate cancer), but not effective in delivering siRNA to suspension cell lines such as Molm13 (AML). See formulation 123A in FIG. 5(a,b).

To achieve efficient delivery to the Molm13 cell line and other suspension cell lines, a compound of formula I such as those disclosed in Examples 2-4 needs to be added the above mentioned ethanolic lipid composition. Optimization studies in the Molm13 cell line suggest that LNPs containing 1-30 mole % of a compound of formula I facilitate siRNA delivery when compared to LNPs that lack a compound of formula I. See FIGS. 1, 4, 5(a,b), and 7

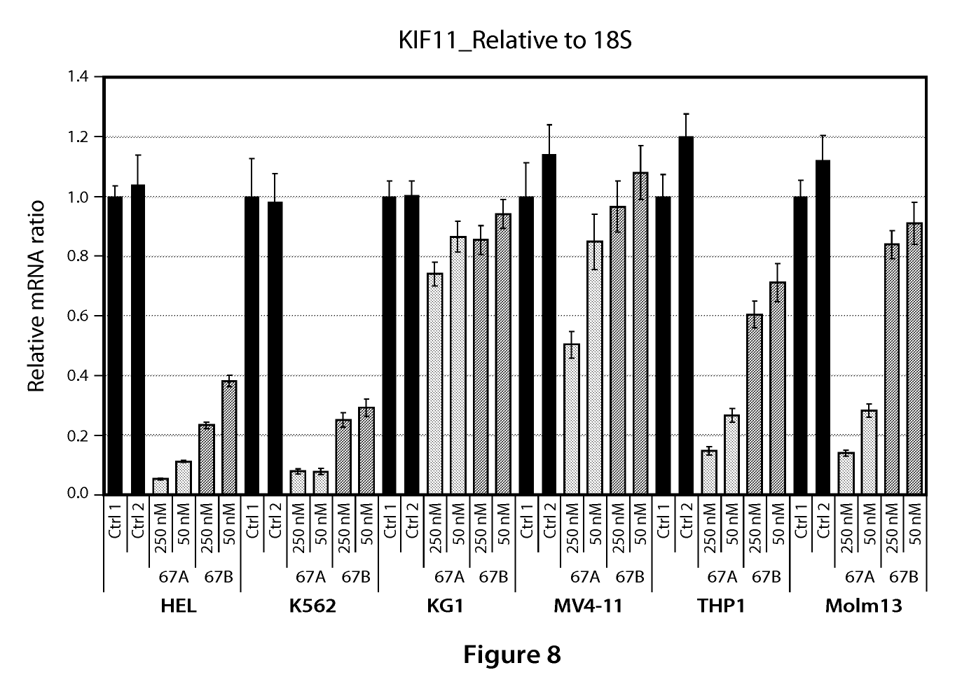

Transfection of several tumor derived leukemia cell lines with siRNAs directed towards KIF11 mRNA produced potent mRNA knockdown shown in FIG. 8. This delivery appears to be dependent on the expression of endocytosis specific genes, Cavl, Rab13, and Rab7B.

Table 3 shows the results from a comparative affymetrix microarray analysis of: (1) THP-1 and Molm13 cell lines in which mRNA knockdown appears to be sensitive to LNP formulations containing a compound of example 4 (DCM-XTC2); as compared to (2) KG1 and MV4-11 cell lines in which mRNA knockdown is less sensitive to LNP formulations containing a compound of example 4 (DCM-XTC2). As indicated, cell lines containing a higher expression level of the listed endocytosis-related genes (THP-1 and Molm13 cell lines) appear to be involved in more efficient DCM-XTC2-mediated siRNA delivery.

TABLE 3
Comparison of gene expression in [KG1 & MV4-11]
vs. [Molm13 & THP1] cells.

| | | | p value |
|---|---|---|---|
| | | fold | of fold |
| gene title | Gene symbol | change | change |
| RAB13, member RAS oncogene family | RAB13 | −52.21 | 0.03 |
| caveolin 1, caveolae protein, 22 kDa | CAV1 | −34.04 | 0.04 |
| RAB7B, member RAS oncogene family | RAB7B | −15.77 | 0.02 |
| RAB7B, member RAS oncogene family | RAB7B | −6.25 | 0.03 |
| caveolin 2 | CAV2 | −6.33 | 0.04 |
| RAB11 family interacting protein 1 | RAB11FIP1 | −2.31 | 0.00 |
| (class I) | | | |
| RAB11 family interacting protein 1 | RAB11FIP1 | −2.02 | 0.01 |
| (class I) | | | |
| RAB11 family interacting protein 1 | RAB11FIP1 | −1.43 | 0.01 |
| (class I) | | | |
| RAB7, member RAS oncogene family- | RAB7L1 | −2.19 | 0.02 |
| like 1 | | | |

Comparative affymetrix microarray analysis of: (1) THP-1 and Molm13 cell lines in which mRNA knockdown appears to be sensitive to LNP formulations containing a compound of example 4 (DCM-XTC2); as compared to (2) KG1 and MV4-11 cell lines in which mRNA knockdown is less sensitive to LNP formulations containing a compound of example 4 (DCM-XTC2). As indicated, cell lines containing a higher expression level of the listed endocytosis-related genes (THP-1 and Molm13 cell lines) appear to be involved in more efficient DCM-XTC2-mediated siRNA delivery.

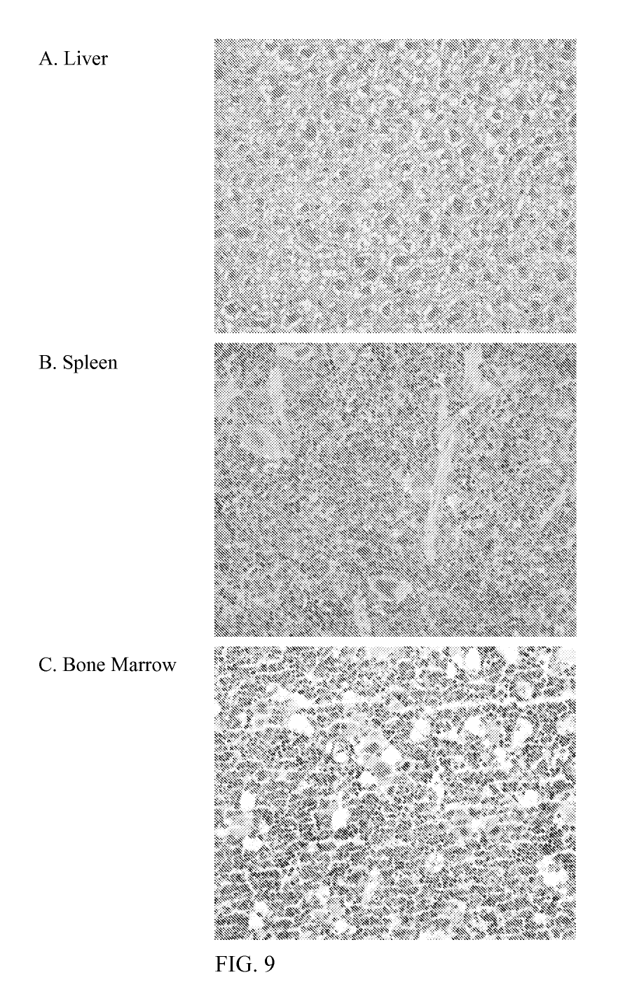

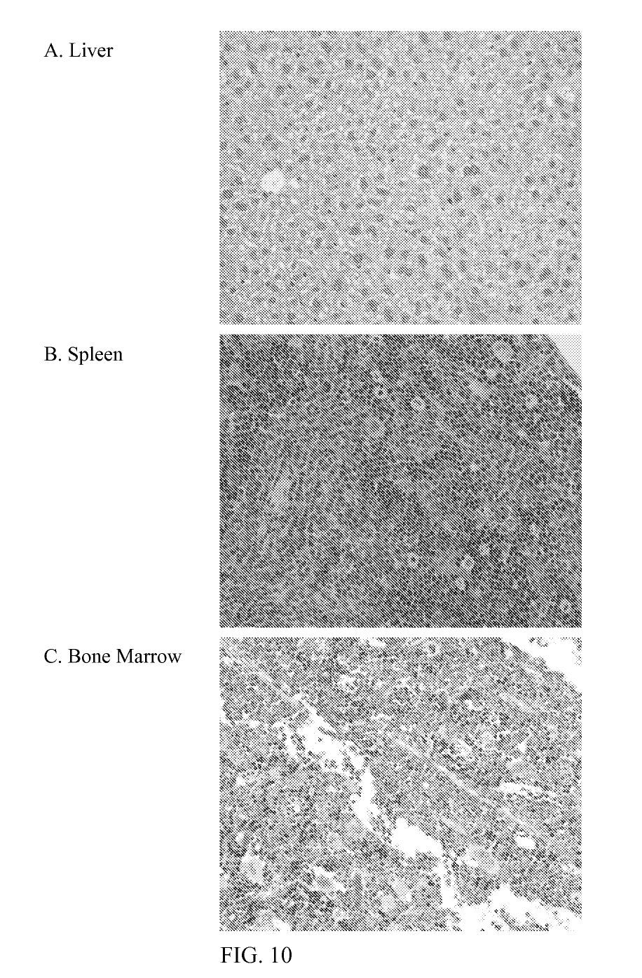

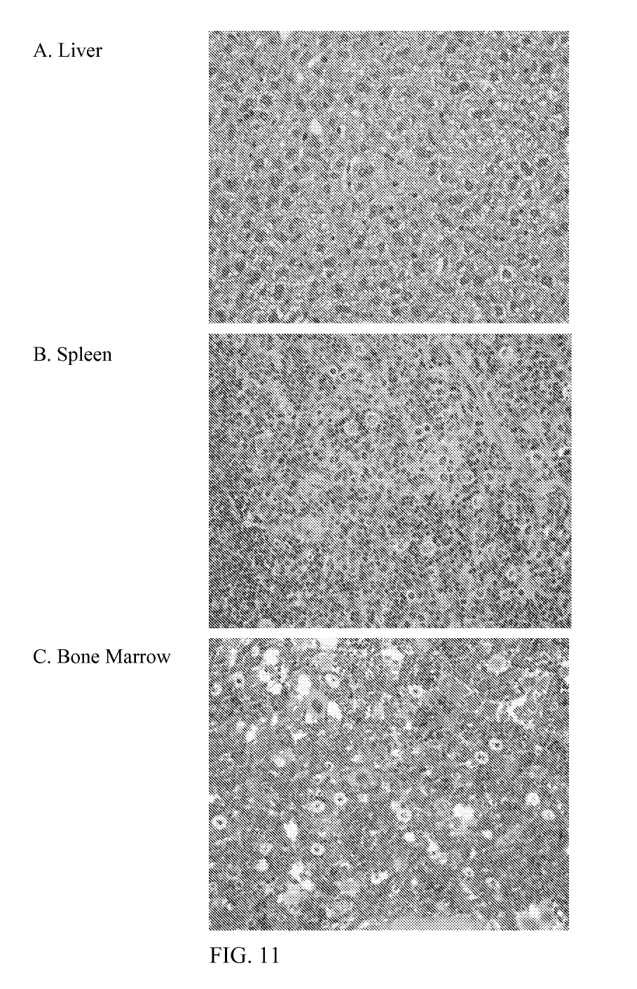

In Vivo Administration. Selected LNPs were dosed IV via tail vein injection on three successive days and the indicated tissues (see FIG. 11-13) were harvested and fixed in 10% zinc-formalin overnight, processed, paraffin embedded, sectioned at 5 μm, and stained with hematoxylin and eosin for histopathology assessment. In vivo delivery and Kif11 knockdown was shown with an increase in aberrant mitotic figures as a surrogate for Kif11 mRNA loss in murine liver, spleen and bone marrow tissue. Mitotic figures refer to chromosome aggregation during mitosis. See FIG. 9-11.

TABLE 4
Effect of siRNA KIF11 in different delivery systems on the
average number of aberrant mitotic figures in bone marrow
Ψ: quantification of mitotic spindles in mouse bone marrows
treated with different siRNA-containing LNP formulations.

| | | | | Average no. of aberrant |
|---|---|---|---|---|
| group | siRNA | Formulation | n | mitotic figures |
| 100 | none | — | 6 | 0 |
| 200 | LUC | — | 6 | 0 |
| 300 | KIF11 | 91D | 6 | 8 |
| 400 | KIF11 | 91E | 6 | 23 |
| 500 | KIF11 | 91G | 6 | 24 |
| 600 | KIF11 | 81F | 6 | 10 |

Ψ = Average number of aberrant mitotic figures in the bone marrow as assessed from 5 consecutive high power (400×) microscopic fields per sample.

1. A lipid nanoparticle composition comprising:
a) a compound of formula I: 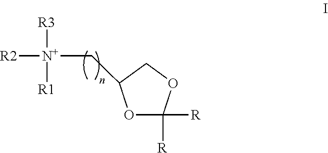
or a pharmaceutically acceptable salt thereof, wherein:
R¹, R², and R³ are independently selected from the group consisting of:
hydrogen,
lower alkyl which is optionally substituted by hydroxy, lower alkoxy or halogen,
lower alkenyl, and
lower alkynyl,
with the proviso that when at least one of R¹, R², or R³ is hydrogen, then at least one of R¹, R², or R³ is: (i) lower alkyl substituted by hydroxy, lower alkoxy or halogen, (ii) lower alkenyl, or (iii) lower alkynyl;
R is selected from the group consisting of:
alkyl having 9 to 22 carbon atoms,
alkenyl having 9 to 22 carbon atoms, and
alkynyl having 9 to 22 carbon atoms; and
n is 1-10;
b) a phospholipid;
c) cholesterol;
d) a pegylated lipid; and
e) a polynucleotide.

2. A lipid nanoparticle composition of claim 1 comprising a compound of formula I, wherein R is linoleyl, R¹ is chloromethyl; R² and R³ are methyl; and n is 2.

3. A lipid nanoparticle composition of claim 1 comprising:
a) a compound of formula I in claim 1;
b) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine;
c) cholesterol;
d) a pegylated lipid compound selected from the group consisting of:
R-3-[(ω-methoxy-poly(ethyleneglycol)₂₀₀₀)carbamoyl]-1,2-dimyristyloxy-propyl-3-amine; and
1,2-distearoyl-sn-glycero-3-phosphoethanolamine-N-[methoxy(polyethylene glycol)-2000]; and
e) a polynucleotide.

4. A lipid nanoparticle composition of claim 1 comprising:
a) a compound of formula I in claim 1 selected from the group consisting of
N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethyl-ethanamonium iodide; and
2-(2,2-di((9Z,12Z)-octadeca-9,12-dienyl)-1,3-dioxolan-4-yl)-N-ethyl-N,N-dimethyl-ethanamonium iodide;
b) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine;
c) cholesterol;
d) a pegylated lipid compound selected from the group consisting of:
R-3-[(ω-methoxy-poly(ethyleneglycol)₂₀₀₀)carbamoyl]-1,2-dimyristyloxy-propyl-3-amine; and
1,2-distearoyl-sn-glycero-3-phosphoethanolamine-N4methoxy(polyethylene glycol)-2000]; and
e) a polynucleotide.

5. A lipid nanoparticle composition of claim 4 further comprising,
2-(2,2-di((9Z,12Z)-octadeca-9,12-dienyl)-1,3-dioxolan-4-yl)-N,N-dimethylethanamine.

6. A lipid nanoparticle composition of claim 4 further comprising hyaluronan.

7. A lipid nanoparticle composition of claim 4, wherein the compound of formula I is N-(chloromethyl)-2-(2,2-di((9Z,12Z)-octadeca-9,12-dienyl)-1,3-dioxolan-4-yl)-N,N-dimethylethanamonium chloride.

8. A lipid nanoparticle composition of claim 7, wherein the phospholipid is 1,2-dipalmitoyl-sn-glycero-3-phosphocholine.

9. A lipid nanoparticle composition of claim 7, wherein the phospholipid is 1,2-distearoyl-sn-glycero-3-phosphocholine.

10. A lipid nanoparticle composition of claim 7, wherein the phospholipid is 1,2-dioleoyl-sn-glycero-3-phosphocholine.

11. A lipid nanoparticle composition of claim 7, wherein the pegylated lipid compound is R-3-[(ω-methoxy-poly(ethyleneglycol)₂₀₀₀)carbamoyl]-1,2-dimyristyloxy-propyl-3-amine.

12. A lipid nanoparticle composition of claim 7, wherein the pegylated lipid compound is 1,2-distearoyl-sn-glycero-3-phosphoethanolamine-N4methoxy(polyethylene glycol)-2000].

13. A lipid nanoparticle composition of claim 7, wherein the polynucleotide is a siRNA molecule which inhibits the expression of KIF11.

14. A lipid nanoparticle composition of claim 1 comprising:
a) a compound of formula I in claim 1 selected from the group consisting of:
N-(chloromethyl)-2-(2,2-di((9Z,12Z)-octadeca-9,12-dienyl)-1,3-dioxolan-4-yl)-N,N-dimethylethanamonium chloride;
2-(2,2-di((9Z,12Z)-octadeca-9,12-dienyl)-1,3-dioxolan-4-yl)-N,N,N-trimethyl-ethanamonium iodide; and
2-(2,2-di((9Z,12Z)-octadeca-9,12-dienyl)-1,3-dioxolan-4-yl)-N-ethyl-N,N-dimethyl-ethanamonium iodide;
b) a phospholipid selected from the group consisting of:
1,2-dipalmitoyl-sn-glycero-3-phosphocholine,
1,2-distearoyl-sn-glycero-3-phosphocholine, and
1,2-dioleoyl-sn-glycero-3-phosphocholine;
c) cholesterol;
d) a pegylated lipid compound selected from the group consisting of:
R-3-[(ω-methoxy-poly(ethyleneglycol)₂₀₀₀)carbamoyl]-1,2-dimyristyloxy-propyl-3-amine; and
1,2-distearoyl-sn-glycero-3-phosphoethanolamine-N-[methoxy(polyethylene glycol)-2000]; and
e) a polynucleotide.

15. A lipid nanoparticle composition of claim 1 comprising:
a) N-(chloromethyl)-2-(2,2-di((9Z,12Z)-octadeca-9,12-dienyl)-1,3-dioxolan-4-yl)-N,N-dimethylethanamonium chloride;
b) 1,2-dipalmitoyl-sn-glycero-3-phosphocholine;
c) cholesterol;
d) a R-3-[(ω-methoxy-poly(ethyleneglycol)₂₀₀₀)carbamoyl]-1,2-dimyristyloxy-propyl-3-amine; and
e) siRNA.

16. A lipid nanoparticle composition of claim 1 comprising:
a) 2-(2,2-di((9Z,12Z)-octadeca-9,12-dienyl)-1,3-dioxolan-4-yl)-N,N-dimethylethanamine;
b) N-(chloromethyl)-2-(2,2-di((9Z,12Z)-octadeca-9,12-dienyl)-1,3-dioxolan-4-yl)-N,N-dimethylethanamonium chloride;
c) 1,2-dipalmitoyl-sn-glycero-3-phosphocholine;
d) cholesterol;
e) a R-3-[(ω-methoxy-poly(ethyleneglycol₂₀₀₀)carbamoyl]-1,2-dimyristyloxy-propyl-3-amine; and
f) siRNA.

17. A lipid nanoparticle composition of claim 1 comprising:
a) 2-(2,2-di((9Z,12Z)-octadeca-9,12-dienyl)-1,3-dioxolan-4-yl)-N,N-dimethylethanamine;
b) 2-(2,2-di((9Z,12Z)-octadeca-9,12-dienyl)-1,3-dioxolan-4-yl)-N,N,N-trimethyl-ethanamonium iodide;
c) 1,2-dipalmitoyl-sn-glycero-3-phosphocholine;
d) cholesterol;
e) a R-3-[(ω-methoxy-poly(ethyleneglycol)₂₀₀₀)carbamoyl]-1,2-dimyristyloxy-propyl-3-amine; and
f) siRNA.

18. A lipid nanoparticle composition of claim 1 comprising:
a) 2-(2,2-di((9Z,12Z)-octadeca-9,12-dienyl)-1,3-dioxolan-4-yl)-N,N-dimethylethanamine;
b) 2-(2,2-di((9Z,12Z)-octadeca-9,12-dienyl)-1,3-dioxolan-4-yl)-N-ethyl-N,N-dimethyl-ethanamonium iodide;
c) 1,2-dipalmitoyl-sn-glycero-3-phosphocholine;
d) cholesterol;
e) a R-3-[(ω-methoxy-poly(ethyleneglycol)₂₀₀₀)carbamoyl]-1,2-dimyristyloxy-propyl-3-amine; and
f) siRNA.

19. A lipid nanoparticle composition of claim 16 wherein:
a) the molar ratio of 2-(2,2-di((9Z,12Z)-octadeca-9,12-dienyl)-1,3-dioxolan-4-yl)-N,N-dimethylethanamine is in the range of about 45-55;
b) the molar ratio of N-(chloromethyl)-2-(2,2-di((9Z,12Z)-octadeca-9,12-dienyl)-1,3-dioxolan-4-yl)-N,N-dimethylethanamonium chloride is in the range of about 1.5-9;
c) the molar ratio of 1,2-dipalmitoyl-sn-glycero-3-phosphocholine is in the range of about 6-8;
d) the molar ratio of cholesterol is in the range of about 33-35; and
e) the molar ratio of a R-3-[(ω-methoxy-poly(ethyleneglycol)₂₀₀₀)carbamoyl]-1,2-dimyristyloxy-propyl-3-amine is in the range of about 0.5-6.

20. A lipid nanoparticle composition of claim 16 wherein:
a) the molar ratio of 2-(2,2-di((9Z,12Z)-octadeca-9,12-dienyl)-1,3-dioxolan-4-yl)-N,N-dimethylethanamine is in the range of about 50-55;
b) the molar ratio of N-(chloromethyl)-2-(2,2-di((9Z,12Z)-octadeca-9,12-dienyl)-1,3-dioxolan-4-yl)-N,N-dimethylethanamonium chloride is in the range of about 2-5;
c) the molar ratio of 1,2-dipalmitoyl-sn-glycero-3-phosphocholine is in the range of about 6.9-7.2;
d) the molar ratio of cholesterol is in the range of about 33-34; and
e) the molar ratio of a R-3-[(ω-methoxy-poly(ethyleneglycol)₂₀₀₀)carbamoyl]-1,2-dimyristyloxy-propyl-3-amine is in the range of about 5-6.
